(19) United States
(12) Patent Application Publication
(10) Pub. No.: US 2026/0241036 A1
(43) Pub. Date: Aug. 20, 2026

(54) CHEMOGENETICS-BASED THERAPY FOR NEUROLOGICAL DISORDERS

(71) Applicant: GATEWAY BIOTECHNOLOGY, INC., St. Louis, MO (US)

(72) Inventors: Jianxin BAO, St. Louis, MO (US); Joseph PINKL, St. Louis, MO (US)

(21) Appl. No.: 19/159,503

(22) PCT Filed: Feb. 29, 2024
(86) PCT No.: PCT/US2024/017872

§ 371 Date: Aug. 25, 2025

Related U.S. Application Data
(60) Provisional application No. 63/487,842, filed on Mar. 1, 2023.

(51) Int. Cl. A61K 48/00; A61K 31/65; A61K 38/17; A61K 45/06; A61P 27/16

(52) U.S. Cl. CPC A61K 48/0058; A61K 31/65; A61K 38/177; A61K 45/06; A61K 48/0033; A61P 27/16

(57) ABSTRACT

The present disclosure provides methods and compositions for the treatment of neurological disease. The methods and compositions include methods and compositions for chemogenetics. The methods include methods for genetically engineering neurons and treating neurological diseases. The compositions include modified proteins and/or nucleic acids for administration to neurons.

### CROSS REFERENCE

This application is a national stage application of International Application No. PCT/US2024/017872, filed Feb. 29, 2024, which claims the benefit of U.S. Provisional Patent Application No. 63/487,842, filed on Mar. 1, 2023, The entirety of all the aforementioned applications is incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 18, 2025, is named “2047-003 NATL SEQLIST.xml” and is 23,540 bytes in size. The sequence listing contained in this .XML file is part of the specification and is hereby incorporated by reference herein in its entirety.

### BACKGROUND OF THE DISCLOSURE

Neurological diseases and conditions, such as auditory diseases and conditions, affect many people worldwide. Chronic auditory diseases and conditions, such as chronic tinnitus, often lead to psychological comorbidities including depression, insomnia, anxiety, and suicidal ideation. Currently, there are no FDA approved drugs to treat or prevent tinnitus. Therapeutic treatments for auditory diseases and disorders are often ineffective because they lack cell-type specificity and/or the ability to effectively target neurons. Thus, novel and effective therapeutics are needed for the improved treatment of these diseases and/or disorders.

### SUMMARY OF THE DISCLOSURE

Disclosed herein are compositions and methods for the treatment of neurological diseases disorders and/or conditions.

One or more aspects, disclosed herein comprises a composition for treating a neurological disease or condition, the composition comprising: a vector, the vector comprising an adeno-associated virus (AAV) and a neuron specific promoter operatively coupled to a gene of interest.

One or more aspects, disclosed herein comprises a composition for treating a neurological disease or condition, the composition comprising: a first vector comprising a first AAV, and a first neuron specific promoter operatively coupled to a first gene of interest, and a second vector comprising a second AAV, and a second neuron specific promoter operatively coupled to a second gene of interest.

One or more aspects, disclosed herein comprises a composition comprising: a vector comprising a gene therapy construct, wherein the gene therapy construct comprises a heterologous nucleic acid encoding a designer receptor, wherein the designer receptor is configured to interact with a small molecule actuator, wherein the vector comprising the heterologous nucleic acid is configured to be expressed in a neuron, wherein expression of the heterologous nucleic acid modulates an inhibitory or an excitatory activity of the neuron, wherein the modulation results in a reduction of a symptom or presence of a disease or condition associated with the neuron.

One or more aspects, disclosed herein comprises a composition comprising a neuron, wherein the neuron comprises a heterologous nucleic acid encoding a protein configured to interact with a small molecule actuator, wherein the heterologous nucleic acid is configured to be expressed in a subject, wherein expression of the heterologous nucleic acid results in a reduction of a symptom of a neurological disease or condition when a small molecule actuator is administered to the subject.

In some embodiments, the first or second vector comprises a nanoparticle.

In some embodiments, the neuron comprises a neuron of the auditory cortex, inferior colliculus, hippocampus, medial geniculate body, amygdala, and/or caudate nucleus.

In some embodiments, the neuron comprises a striatal neuron.

In some embodiments, the striatal neuron comprises a neuron of the caudate nucleus, the putamen, the nucleus accumbent, or a combination thereof.

In some embodiments, the striatal neuron comprises a neuron of the left caudate nucleus, the right caudate nucleus, or the left caudate nucleus and the right caudate nucleus.

In some embodiments, the neuron comprises an excitatory and/or inhibitory neuron.

In some embodiments, the neuron comprises a GABAergic neuron or a glutamatergic neuron.

In some embodiments, wherein the heterologous nucleic acid comprises one or more sequences configured to direct integration of the heterologous nucleic acid into a genomic location of a mammalian cell.

In some embodiments, the heterologous nucleic acid comprises a deoxyribonucleic acid (DNA).

In some embodiments, the heterologous nucleic acid comprises a ribonucleic acid (RNA).

In some embodiments, the heterologous nucleic acid comprises a nucleotide sequence comprising a promoter operatively coupled to an open reading frame of a gene of interest.

In some embodiments, the open reading frame of the gene of interest encodes a polypeptide.

In some embodiments, the promoter comprises a neuron specific promoter.

In some embodiments, the neuron specific promoter comprises a CamKII promoter, an hSYN promoter, or a Vgat promoter.

In some embodiments, the promoter comprises a bacterial PA, PR, and/or PR1/PR2 promoter.

In some embodiments, the gene of interest encodes a modified neuronal receptor.

In some embodiments, the gene of interest encodes a modified muscarinic receptor

In some embodiments, the gene of interest encodes a modified human muscarinic receptor.

In some embodiments, the modified muscarinic receptor comprises a designer receptor exclusively activated by designer drugs (DREADD).

In some embodiments, the DREADD comprises one or more of hM3Ds, hM1Dq, hMD5q, hM4Di, or hM2Di, or hM3Dq.

In some embodiments, the DREADD comprises an amino acid sequence exhibiting at least about 90%, 95%, 97%, 98%, 99% identity to or is identical to SEQ ID NO: 1.

In some embodiments, the heterologous gene of interest is delivered to the cell using an adeno-associated virus (AAV).

In some embodiments, the AAV comprises AAV1, AAV2, AAV5, AAV8, AAV9 or AAV-DJ.

In some embodiments, the neurological disease or condition comprises an neurological disease or condition, such as tinnitus.

In some embodiments, the small molecule actuator comprises an atypical antipsychotic.

In some embodiments, the atypical antipsychotic comprises Clozapine-N-oxide.

In some embodiments, the symptom of the neurological disease or condition comprises vertigo, dizziness, hearing impairment, buzzing, clicking, humming, hissing, roaring, or ringing or a combination thereof.

In some embodiments, the neurological disease or condition comprises tinnitus, Alzheimer's disease, Meniere's disease, noise induced hearing loss (NIHL), sensorineural hearing loss, auditory neuropathy spectrum disorder, and/or central auditory processing disorder.

One or more aspects, disclosed herein comprises a pharmaceutical composition comprising a heterologous nucleic acid and a pharmaceutically acceptable, carrier, excipient, and/or diluent.

In some embodiments, the pharmaceutical composition is formulated for delivery by direct injection to a brain of the subject.

In some embodiments, the pharmaceutical composition is formulated for delivery by nasal or cochlear injection to the subject.

In some embodiments, the pharmaceutical formulation is formulated for delivery by lipid nanoparticles, liposomes, electroporation, plasmids, or non-viral delivery methods.

In some embodiments, the non-viral delivery methods comprise, lipofection, nucleofection, microinjection, biolistics, virosomes, liposomes, immunoliposomes, polycation or lipid: nucleic acid conjugates, naked DNA, artificial virions, or agent-enhanced uptake of DNA.

One or more aspects, disclosed herein comprises a method of genetically engineering or modulating a neuron of a subject comprising administering a heterologous nucleic acid to the subject thereby genetically engineering the neuron.

In some embodiments, the neuron comprises a neuron of the auditory cortex, inferior colliculus, hippocampus, medial geniculate body, amygdala, and/or caudate nucleus.

In some embodiments, the neuron comprises a striatal neuron.

In some embodiments, the striatal neuron comprises a neuron of the caudate nucleus, the putamen, the nucleus accumbent, or a combination thereof.

In some embodiments, the striatal neuron comprises a neuron of the left caudate nucleus, the right caudate nucleus, or the left caudate nucleus and the right caudate nucleus.

In some embodiments, the neuron comprises a medium-sized GABAergic spiny projection neuron or a glutamatergic neuron.

In some embodiments, the method of genetically engineering the cell or treating the subject comprises administering the heterologous nucleic acid comprises direct injection of the heterologous nucleic acid to the brain of a subject.

In some embodiments, the method of genetically engineering the cell or treating the subject comprises administering the heterologous nucleic acid comprises nasal and/or cochlear injection.

In some embodiments, the method of genetically engineering the cell or treating the subject comprises administering the heterologous nucleic acid comprises administering the heterologous nucleic acid using lipid nanoparticles, liposomes, electroporation, plasmids, or non-viral delivery methods to a subject.

In some embodiments, the non-viral delivery methods comprise lipofection, nucleofection, microinjection, biolistics, virosomes, liposomes, immunoliposomes, polycation or lipid: nucleic acid conjugates, naked DNA, artificial virions, or agent-enhanced uptake of DNA.

In some embodiments, the heterologous nucleic acid is administered at a dose of from about 1 nl to about 1000 nl.

In some embodiments, the heterologous nucleic acid is administered at a viral titer of from about 1×10¹⁴ vg/kg to about 3×10¹⁴ vg/kg.

In some embodiments, genetically engineering the neuron comprises expressing a heterologous G-protein coupled receptor (GPCR) in the neuron.

In some embodiments, the heterologous GPCR comprises hM3Dq.

One or more aspects, disclosed herein comprises a method of treating a subject with a neurological disease or condition comprising administering the heterologous nucleic acid of any one of the preceding claims or the pharmaceutical composition of any one of the preceding claims to a neuron of the subject with the disease or condition, expressing the heterologous nucleic acid as a protein configured to interact with a small molecule actuator, and activating the protein with the small molecule actuator, thereby treating the neurological disease and/or condition.

In some embodiments, the small molecule actuator comprises an atypical antipsychotic.

In some embodiments, the atypical antipsychotic comprises Clozapine-N-oxide.

In some embodiments, the neurological disease or condition comprises an auditory disease or condition.

In some embodiments, wherein the auditory disease or condition comprises tinnitus, Meniere's disease, noise induced hearing loss (NIHL), sensorineural hearing loss, auditory neuropathy spectrum disorder, and/or central auditory processing disorder.

In some embodiments, the subject comprises a mammal.

In some embodiments, the subject comprises a human.

In some embodiments, the heterologous nucleic acid is administered to a neuron of the auditory cortex, inferior colliculus, hippocampus, medial geniculate body, amygdala, and/or caudate nucleus.

In some embodiments, the heterologous nucleic acid is administered to the neurons of the left caudate nucleus, the right caudate nucleus, or the right and left caudate nucleus.

In some embodiments, the small molecule actuator is administered to the subject by injection.

In some embodiments, the small molecule actuator is administered to the subject by intraperitoneal injection.

In some embodiments, the small molecule actuator is administered to the subject at a dose of from about 0.5 mg/kg to about 20 mg/kg.

In some embodiments, the small molecule actuator is administered to the subject once per day, twice per day, three times per day, four times per day, or more than four times per day.

In some embodiments, the small molecule actuator is administered to the subject every 2 hours, every 3 hours, or every four hours.

In some embodiments, wherein the small molecule actuator is administered to the subject once per day, twice per day, or more than twice per day for at least one day, at least two days, at least 3 days, at least one week, at least two weeks, at least one month, or at least more than one month.

One or more aspects, disclosed herein comprises a method of treating a subject with a disease or condition comprising activating inhibitory or excitatory neurons in a brain region wherein the activating comprises administering the heterologous nucleic acid of any one of the preceding claims to an inhibitory or excitatory neuron within the brain region, expressing the protein encoded by the heterologous nucleic acid and activating the protein, thereby treating the disease or condition.

In some embodiments, the brain region comprises the auditory cortex, inferior colliculus, hippocampus, medial geniculate body, amygdala, and/or caudate nucleus.

In some embodiments, the inhibitory or excitatory striatal neuron comprises an inhibitory neuron of the left caudate nucleus, the right caudate nucleus, or the left caudate nucleus and the right caudate nucleus.

In some embodiments, the inhibitory or excitatory neuron comprises a GABAergic neuron a glutamatergic neuron, a dopaminergic neuron, or a serotonin neuron.

In some embodiments, the inhibitory neuron comprises a medium-sized GABAergic spiny projection neuron.

In some embodiments, the neurological disease or condition comprises an auditory disease or condition.

In some embodiments, the auditory disease or condition comprises tinnitus, Meniere's disease, noise induced hearing loss (NIHL), sensorineural hearing loss, auditory neuropathy spectrum disorder, and/or central auditory processing disorder.

In some embodiments, the heterologous nucleic acid comprises an inhibitory or excitatory DREADD.

In some embodiments, the DREADD comprises the heterologous nucleic acid of any one of the preceding claims wherein the neuron specific promoter comprises a CamKII promoter, an hSYN promoter, or a Vgat promoter.

One or more aspects, disclosed herein comprises a method of activating the protein of any one of the preceding claims wherein the activating comprises administering an atypical antipsychotic to a subject.

In some embodiments, the atypical antipsychotic comprises Clozapine-N-oxide.

In some embodiments, at least one additional composition or pharmaceutical composition can be administered to a subject.

In some embodiments, the at least one additional composition or pharmaceutical composition comprises a therapeutic agent.

In some embodiments, the therapeutic agent comprises a tetracycline promoter and tetracycline.

### INCORPORATION BY REFERENCE

All publications, patents, and patent applications mentioned in this specification are herein incorporated by reference to the same extent as if each subject publication, patent, or patent application was specifically indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A depicts the experimental design of a sound-based avoidance detection (SBAD) model. FIG. 1B depicts the SBAD Go Trial set up. FIG. 1C depicts the SBAD No-Go Trial set up.

FIG. 2A depicts a three-dimensional rendering of a mouse brain with the chemogenetic target 205 highlighted at different perspectives. FIG. 2B depicts the results of No-Go and Go SBAD training (days 1-15 indicated numerically) and test results of three post-operative conditions (B1-B3: baseline, +: 1 hour post CNO IP injection,—: 1 hour post saline IP injection) completed three times each for each animal. FIG. 2C depicts SBAD test scores of three post-operative conditions were averaged for each animal (colored markers) with total averages between all animals. FIG. 2D depicts No-Go scores for the Clozapine-N-oxide (CNO) condition for each injected AAV dose (50 nL vs 150 nL). FIG. 2E depicts statistical analysis results of the Go and No-Go testing.

FIG. 3A depicts a longitudinal cross-sectional perspective of the target injection coordinates for the Inferior Colliculus (IC). FIG. 3B depicts a lateral cross-sectional perspective of the target injection coordinates for the CIC

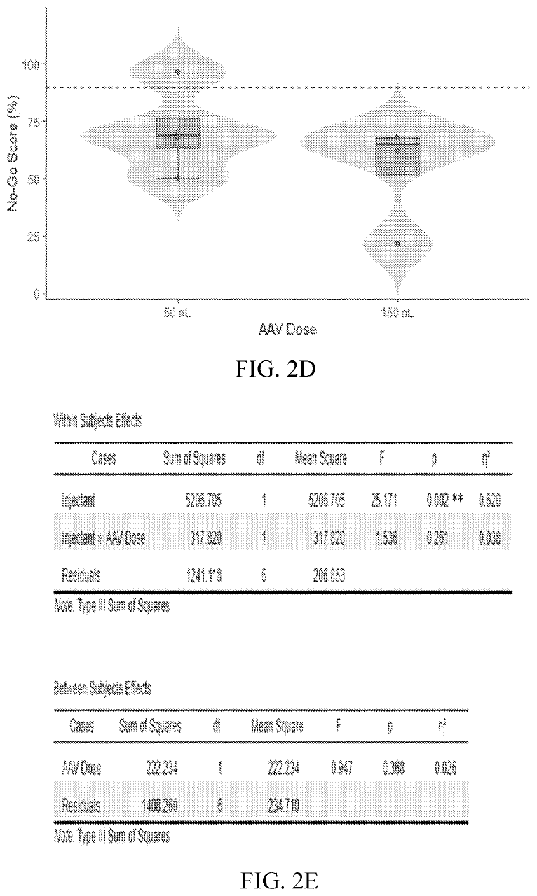

FIG. 4 depicts the IC and Caudate Nucleus (CN) target areas in a mouse brain relative to bregma and lambda.

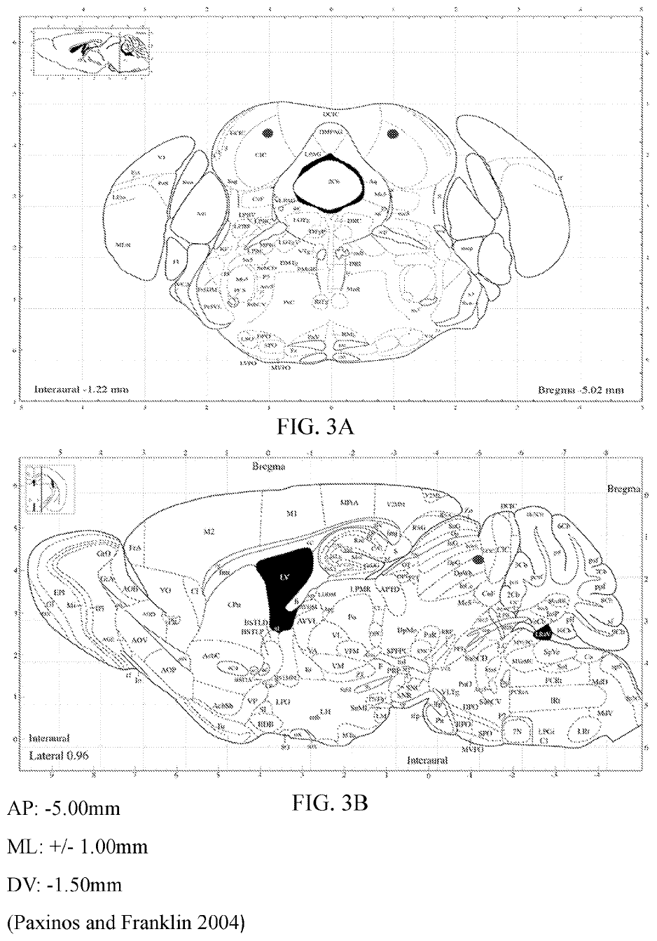

FIG. 5A depicts a stereotaxic micro injection head frame set up. FIG. 5B depicts MRI image of general target areas of the ICC (510) and CN (515) with specific target sites highlighted.

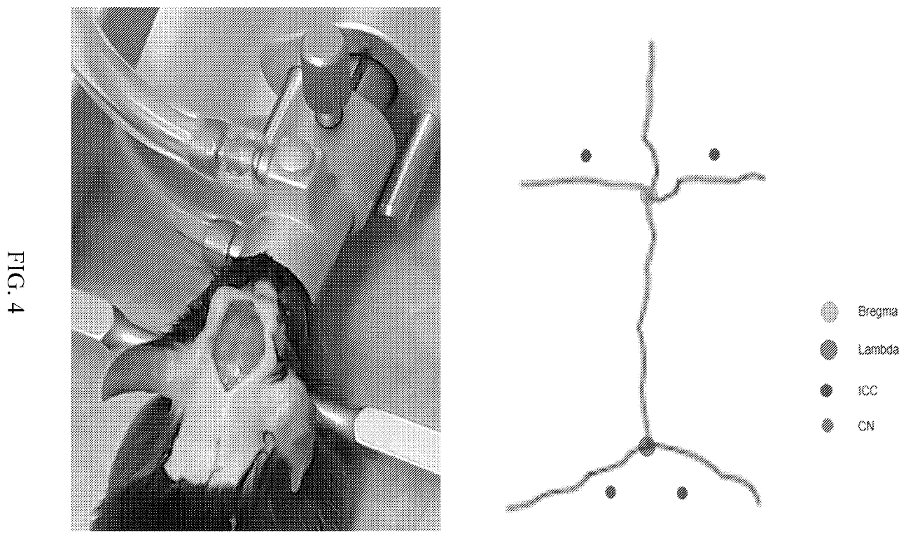

FIG. 5C depicts immunofluorescence staining of glutamatergic neuronal cell bodies and immunolabeled mCherry in an IC sample of a representative experimental animal. FIG. 5D depicts animals receiving intraperitoneal (IP) injections of CNO and saline (5 mg/kg) 1 hour prior to SBAD testing. CNO and saline injections were performed alternatively over 6 consecutive days. FIG. 5E depicts CNO mechanism of action in an AAV injected animal.

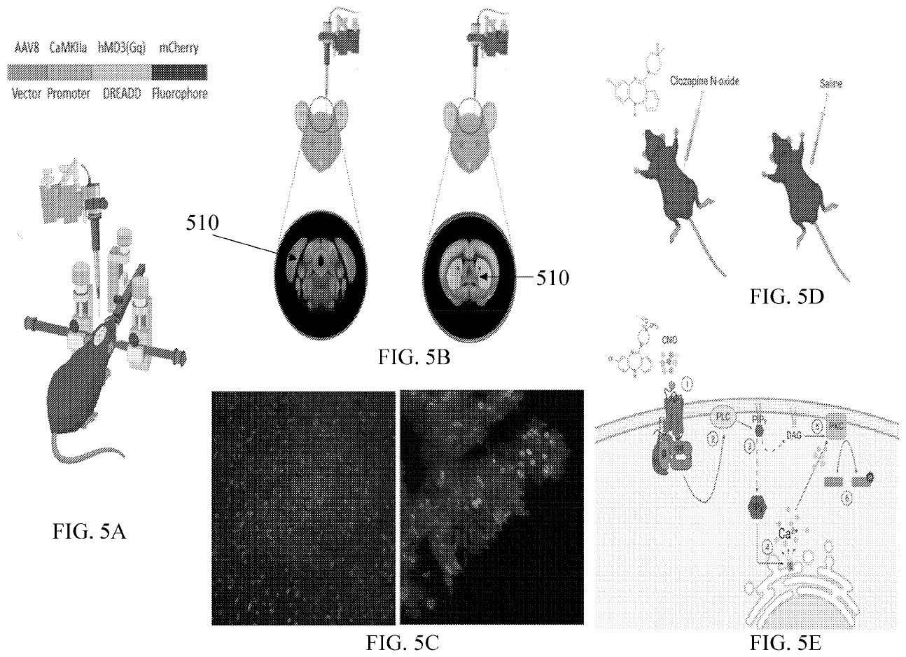

FIG. 6A depicts Three-dimensional rendering of mouse brain with the CN and chemogenetic target highlighted. FIG. 6B depicts No-Go and Go SBAD training (days 1-10 indicated numerically) and test results of three post-operative conditions (B1-B3: baseline, +: 1 hour post CNO IP injection,—: 1 hour post saline IP injection) completed three times each for each animal. FIG. 6C depicts SBAD test scores of three post-operative conditions that were averaged for each animal with total averages between all animals indicated by the black markers.

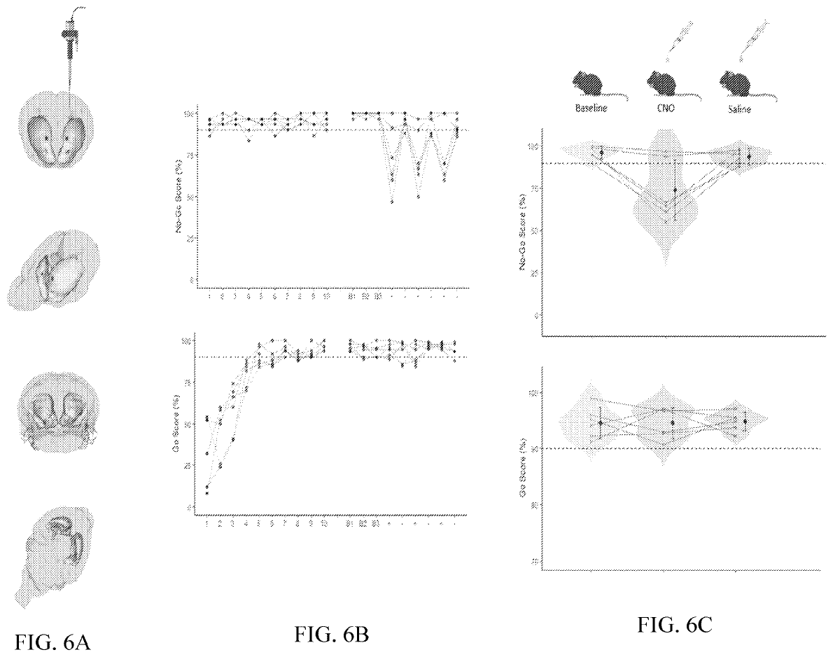

FIG. 7 depicts electrophysiologic measures in mice injected with CNO.

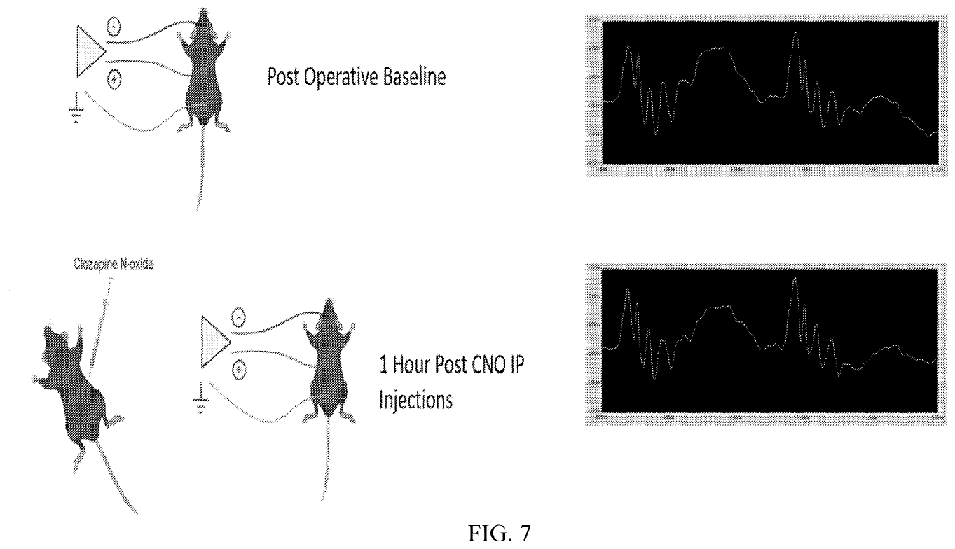

### DETAILED DESCRIPTION OF THE DISCLOSURE

Chemogenetics and chemogenetics-based gene therapy methods, such as compositions and methods comprising Designer Receptors Exclusively Activated by Designer Drugs, (DREADDs), are techniques and methods by which proteins can be engineered to interact with small molecule ligands. These compositions and methods can be used to specifically target cells, including neurons. By using inhibitory and excitatory DREADDs to target neurons, the methods and compositions of the present disclosure can be used to treat neurological diseases or conditions.

### Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some embodiments, the subject is not necessarily diagnosed or suspected of being at high risk for the disease.

As used herein, the term “in vivo” is used to describe an event that takes place in a subject's body.

As used herein, the term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen or method for obtaining beneficial or desired results in the recipient. Beneficial or desired results include but are not limited to a therapeutic benefit and/or a prophylactic benefit. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject can still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

The terms “polypeptide” and “protein” are used interchangeably to refer to a polymer of amino acid residues and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some aspects, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared.

The polypeptides described herein can be encoded by a nucleic acid. In some embodiments, a nucleic acid comprises a polynucleotide comprising two or more nucleotide bases. The terms “nucleic acid” and “nucleic acid molecule” can be used interchangeably. The terms refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single stranded, double-stranded and the like). A nucleic acid can be, or can be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in some embodiments. A nucleic acid in some embodiments can be from a single chromosome (e.g., a nucleic acid sample can be from one chromosome of a sample obtained from a diploid organism). Nucleic acids also include derivatives, variants and analogs of RNA or DNA synthesized, replicated or amplified from single-stranded (“sense” or “antisense”, “plus” strand or “minus” strand, “forward” reading frame or “reverse” reading frame) and double stranded polynucleotides. Deoxyribonucleotides include deoxyadenosine, deoxycytidine, deoxyguanosine and deoxythymidine. For RNA, the base cytosine is replaced with uracil and the sugar 2′ position includes a hydroxyl moiety. A nucleic acid can be prepared using a nucleic acid obtained from a subject as a template. In some embodiments, the nucleic acid comprises a component of a vector that can be used to transfer the polypeptide encoding polynucleotide into a cell. In some embodiments, a heterologous nucleic acid comprises a nucleic acid that is exogenous to a cell or cell population being modified. A heterologous nucleic acid can comprise a gene or nucleotide sequence that is a modified form of an endogenous gene or can comprise a recombinant gene or nucleic acid sequence. Heterologous nucleic acids comprise regulatory sequences, encode fusions to endogenous genes or other modifications that increase the therapeutic or diagnostic potential of a gene or nucleotide sequence. “Heterologous” as used in some embodiments disclosed herein refers to a nucleic acid, gene, polypeptide or protein comprising a nucleic acid, gene, polypeptide or protein that is not a natural component of an adeno-associated viruses (AAVs) or additional vector, or cell described herein. Heterologous nucleic acids can encode a gene or RNA (e.g., antisense or siRNA) not normally expressed by the AAVs, vectors, or cells described herein including synthetic, mammalian, or human genes or RNAs.

As described herein “operatively coupled” refers to the arrangement of a promoter or regulatory region to an open reading frame (e.g., gene of interest or target gene) on a nucleic acid molecule that results in transcription of the open reading frame. Generally, a regulatory region will be 5′ to the open reading frame and can comprise one or more intervening nucleotides that do not significantly inhibit transcription of the open reading frame.

A designer receptor exclusively activated by designer drugs (DREADD) is a class of artificially engineered protein receptors used in the field of chemogenetics which are selectively activated by certain ligands. Non-limiting examples of DREADDs can be found in Urban DJ and Roth BL, 2015, DREADDs (Designer Receptors Exclusively Activated by Designer Drugs): Chemogenetic Tools with Therapeutic Utility, Annu. Rev. Pharmacol. Toxicol. 55:15.1-15.19 and Roth, 2016, DREADDs for Neuroscientists, Neuron. 89:683-694.

As used herein, the term “inhibitory DREADD” refers to an inhibitory class DREADD, such as Gi-GPCR, KORD-GPCR activated, or similar DREADD, or a DREADD that activates an inhibitory neuron. As used herein, the term “excitatory DREADD” refers to an excitatory class of DREADD, such as a Gq-GPCR activate DREADD, or similar DREADD, or a DREADD that activates an excitatory neuron.

As used herein, the terms “homologous,” “homology,” or “percent homology” are used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

As used herein, the term “serotype” refers to a distinguishable strain of a microorganism. A serotype can also refer to a group of organisms that have the same type and number of surface antigens. Serotypes may or may not differ from strains, which are isolates of a single culture. Serotypes may or may not differ from genotypes which have different sets of genes.

The terminology used herein is for describing particular embodiments only and is not intended to be limiting of the disclosure. The term “comprising” and “comprises”, used in the claims, should not be interpreted as being restricted to the components and steps listed thereafter; they do not exclude other components or steps. They need to be interpreted as specifying the presence of the stated features, integers, steps and/or components as referred to, but does not preclude the presence and/or addition of one or more other features, integers, steps or components, or groups thereof. Thus, the scope of the expression “a composition comprising A and B” should not be limited to compositions consisting only of components A and B. Also, the scope of the expression “a method comprising the steps X and Z” should not be limited to methods consisting exclusively of those steps.

As used herein, the term “about” a number refers to that number plus or minus 10% of that number. The term “about” a range refers to that range minus 10% of its lowest value and plus 10% of its greatest value. For example, the term “about” can be immediately understood as within 10%, 9%, 8%, 7%, 6%, 5%, 4%, 3%, 2%, 1%, 0.5%, 0.1%, 0.05%, or 0.01% of the stated value. As an illustration, a numerical range of “about 1 to about 5” should be interpreted to include not only the explicitly recited values of about 1 to about 5, but also include individual values and sub-ranges within the indicated range, which includes, at very least, the degree of expected experimental error, technical error and instrumental error for a given experiment, technique or an instrument used to measure a value.

As used herein, the term “and/or” includes any combinations of one or more of the associated listed items. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it may be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

As used herein, the terms “active agent”, “pharmaceutical active agent”, “active”, “API”, “active pharmaceutical ingredient”, “active substance”, “active molecule”, “active compound” or “drug” can be used interchangeably.

As used herein, the term “gene therapy construct” refers to the composition(s) disclosed herein (e.g., a heterologous nucleic acid encoding a DREADD). In some embodiments, the gene therapy construct is configured to be expressed in a brain region.

The term “condition” indicates a physical status of an individual (as a whole or as one or more of its parts e.g., body systems), that does not conform to a standard physical status associated with a state of complete physical, mental and social well-being for the individual. Conditions herein described comprise disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms in an individual.

### Heterologous Nucleic Acids

In one embodiment, a composition and/or gene therapy construct of the present disclosure further comprises a heterologous nucleic acid (e.g., a DNA or an RNA). In another embodiment, the heterologous nucleic acid can comprise one or more sequences to direct integration into a genomic location of a cell. Some embodiments of the present disclosure comprise a cell. In some embodiments, the cell comprises a bacterial, archaeal, plant, fungal or animal cell. In some embodiments, the animal cell comprises a human cell, amphibian cell, reptilian cell, mammalian cell, avian cell, or fish cell. A mammalian cell comprises any cell from or derived from any mammal (e.g., a human, a hamster, a mouse, a monkey, a rat, a pig, a cow, or a rabbit).

In some embodiments, a heterologous nucleic acid comprises a sequence comprising a promoter. In some embodiments, the heterologous nucleic acid sequence comprises an open reading frame of a gene of interest. In some embodiments, a gene of interest comprises a non-coding region (e.g., a UTR, or a promoter). In some embodiments, the gene of interest comprises a polypeptide. In some embodiments, the heterologous nucleic acid comprises a DREADD. A neuron that has been modified and/or genetically engineered to express a polypeptide, such as a DREADD, using a method and/or composition disclosed herein can be described as expressing a “heterologous nucleic acid.”

In some embodiments, a polypeptide comprises an antibody, a contractile protein, an enzyme, a hormonal protein, a structural protein, a storage protein, a small molecule, or a transport protein. Non-limiting examples of enzymes include hydrolase, isomerases, nucleases, ligases, transferases, and oxidoreductases. In some embodiments, a gene of interest can be a G protein-coupled receptor (GPCR), or a kinase. In some embodiments, a gene of interest comprises a modified muscarinic receptor. In some embodiments, a gene of interest comprises a modified human muscarinic receptor. be a muscarinic receptor. The muscarinic receptor can be a human muscarinic receptor.

In some embodiments, a gene of interest comprises a DREADD. The DREADD can be a Gi-DREADD, a Gq-DREADD, a Gs-DREADD, an Arrestin-DREADD, an Axonally-targeted silencing DREADD, or a KORD-DREADD. (e.g., hM3Dq, hM1Dq, hMD5q, hM4Di, or hM2Di). In some embodiments, the gene of interest comprises about 60%, about 70%, about 80%, about 90%, about 95%, about 99%, about 99.5% or about 100% identity to TTCAAGGCCTGCTCGGGTGCGCGCTTGTGAAAAATGACCGACTGTCTCTGCTGGTACTG CTGCTTGCGCCTCTTTTTTTTGTCACACTGGCACAGCAGCAGCATCTTGAAAGTGGTTCT GAATGTTTTGTTGCACAGAGCATAGCACACGGGGTTCACGGTGCTGTTGATGTAGCAC AGCCAGTAGCCCAGATTCCAAAAGGTTTTGGGTATGCAGCTGTCACAAAAGGTGTTCA CCAGAACCATGATGTTGTATGGGGTCCAAGTGATGATGAAGGCAAGCAAGATCGCACT GAGGGTCTGGGCCGCTTTCTTCTCCTTGACCAGGGACATCCTTTTCCGCTTAGTGATCTG ACTTCTGGTCTTCAGAGCAAACCTCTTGGCCAGAGTGGCTTCCTTGAAGGACAGAGGTA GAGTGGCCGTGCTCTTACCCACTGAGGAGTTGACGTCAGAAGTCTTAGCTGTGTCCACG GCTGACTCTAGCTGGATGGGAAGCTTGGAGAAGCTTTTTGGAAAACTGCCTCCATCGTC CACGCTCTTCTGGGCCTGCAGCTTGTCGGCTTTCCTCTCCAAGTCCACCATCCCCAGCTC CTCCTCAGGCACCTGCAGGTTGTCCGATGAGGGTAACTTGGTGGAGTTGAGGATGGTG CTGTGACCCGGAAGCTTGAGCACGATGGAGTAGATGGCTCTCGTCTCGGAGCCAATGT CCTCCTCGTCGGAGGAGGCGGAGTTCTCCAGGGAGGCAGCAGCATCATTGTTGTTCCA ACTGTCACTGCTGCTGTGGTCTTGGTCCATCTGCTCGGAGCTGGGTTTCCAGCTCTTGGT TGTGAACCAGAAGTGGCAGCGGCCATACTTCCTCCTGTTGGAGCGTTTCATGCTTTGCT GTTGAAGTTCGTAACTGCTGCAGCTTCGAGAACTGCCCGTGGGGTGGACAAAGTTTTCT GTCTCTGCCTCTGTCCCAGAGGCTTGCAGGCCAGCAAGCTCTTTGGTACGCTTTTCAGT TTCCTTATAGATCCTCCAGTATAAAATAGTCATAATGGTGACAGGCATATAAAAACCA GCGATGGCTGTGCCAAAAGTAATGGTGGGCTCACTGAGGAACTGAATGAAGCACTCTC CCGGAGGCACAGTTCTCTTTCCAACAAAGTATTGCCAGAACAAGATGGCAGGAGCCCA AAGGACAAAGGAGATGACCCAAGCCAGACCGATCATCACACCGGCTCTCTTTGTTGTT CGTTTGGCTCGGTACGTGAGCGGCCTCGTGATGGAAAAGTATCTGTCAAAGCTGATGA CCAGAAGATTCATAACAGAGGCATTGCTGGCTACGCAGTCAATGGCAAGCCAGAGGTC ACAGGCCAAGTTCCCTAAGGCCCATCGATTCATGATGATGTAGGTCGTAAACAGATTC ATTGAAATGACCCCGATAATCAGATCGGCACAGGCCAGGCTTAAGAGGAAGTAGTTGT TGACCGTCTTCAGCTGCTTGTTGACCTTAAATGACACAATTACCAGGATGTTGCCGATG ATGGTCACCAAGGCCAGGATGCCCGTTAAGAAAGCGATGAAGACCACTTGCCAGACGG TATGACCTCCCAGAGGGTCATCGGTGGTACCGTCTGGAGAGGAGAAATTGCCAGCTGC TCGAGAAACATTGTAGCTGCCGAAATGAGTGACGGTTCCCGGGGGCAGCCCTGCATCG GAGGGGCTGTGTATCCAGGAGGAGCTGATGTTTGGAAACAAAGGCGAGGTTGTACTGT TATTGTGCAAGGTCATGGTGGC (SEQ ID NO: 1). In some embodiments, the gene of interest consists of about 60%, about 70%, about 80%, about 90%, about 95%, about 99%, about 99.5% or about 100% identity to SEQ ID NO: 1.

In some embodiments, the heterologous nucleic acid comprises an AAV vector, a promoter, and a gene of interest. The AAV vector can be an AAV1, AAV1, AAV2, AAV3, AAV4, AAV5, AAV6, AAV7, AAV8, AAV9, AAV10, AAV11, AAV12, AAV13, Rh10, PHP.B, PHP.eB, or PHP.S as well as variants (e.g., capsid variants, such as amino acid insertions, additions and substitutions) thereof. AAV capsids can include a VP1 protein, a VP2 protein, and/or a VP3 protein wherein VP2 and VP3, can be amino-terminal truncations of VP1.

In some embodiments, the heterologous nucleic acid further comprises a transcriptional activator and/or enhancer.

In some embodiments, the promoter comprises a neuron specific promoter. The promoter can comprise a promoter specific to an inhibitory neuron or specific to an excitatory neuron. The promoter can comprise a CamKII promoter, an hSYN promoter, or a Vgat promoter.

In some embodiments, the gene of interest comprises an open reading frame of a gene of interest. The open reading frame of the gene of interest can encode a polypeptide. The polypeptide can comprise about 60%, about 70%, about 80%, about 90%, about 95%, about 99%, about 99.5% or about 100% identity to the amino acid sequence of SEQ ID NO: 1.

### Promoters

In some embodiments, a composition and/or gene therapy construct of the present disclosure comprises a promoter. In some embodiments, the promoter can be tissue specific. In some embodiments, a promoter can be cell-type specific. Non-limiting examples of cell-type specific promoters include neuron specific promoters, muscle specific promoters, blood cell specific promoters, skin cell specific promoters, endothelial cell specific promoters, or epithelial cell specific promoters.

In some embodiments, the cell-type specific promoter can be neuron specific. As disclosed in some embodiments herein, the neuron specific promoter comprises a promoter that only functions in neurons to turn on and/or off genes that are specific to neurons. In some embodiments, the neuron specific promoter comprises a synapsin I (SYN) promoter (e.g., hSYN1), a calcium/calmodulin-dependent protein kinase II (CamKII) promoter, a vesicular GABA transporter (Vgat) promoter, a tubulin alpha I, a neuron-specific enolase, a platelet-derived growth factor beta chain promoter, an astrocyte-specific glial fibrillary acidic protein (GFAP) promoter, a cerebellar Purkinje cell-specific L7-6 promoter, a dopamine receptor D1 (DRD1) promoter, a dopamine receptor D2 (DRD2) promoter, a parvalbumin (Pvalb) promoter, or a distal-less homeobox (Dlx) promoter.

In some embodiments, the promotor comprises a nucleic acid and/or amino acid sequence that is about 60%, about 70%, about 80%, about 90%, about 95%, about 99%, about 99.5% or about 100% identity to TTAACATTATGGCCTTAGGTCACTTCATCTCCATGGGGTTCTTCTTCTGATTTTCTAGAA AATGAGATGGGGGTGCAGAGAGCTTCCTCAGTGACCTGCCCAGGGTCACATCAGAAAT GTCAGAGCTAGAACTTGAACTCAGATTACTAATCTTAAATTCCATGCCTTGGGGGCATG CAAGTACGATATACAGAAGGAGTGAACTCATTAGGGCAGATGACCAATGAGTTTAGGA AAGAAGAGTCCAGGGCAGGGTACATCTACACCACCCGCCCAGCCCTGGGTGAGTCCAG CCACGTTCACCTCATTATAGTTGCCTCTCTCCAGTCCTACCTTGACGGGAAGCACAAGC AGAAACTGGGACAGGAGCCCCAGGAGACCAAATCTTCATGGTCCCTCTGGGAGGATGG GTGGGGAGAGCTGTGGCAGAGGCCTCAGGAGGGGCCCTGCTGCTCAGTGGTGACAGAT AGGGGTGAGAAAGCAGACAGAGTCATTCCGTCAGCATTCTGGGTCTGTTTGGTACTTCT TCTCACGCTAAGGTGGCGGTGTGATATGCACAATGGCTAAAAAGCAGGGAGAGCTGGA AAGAAACAAGGACAGAGACAGAGGCCAAGTCAACCAGACCAATTCCCAGAGGAAGCA AAGAAACCATTACAGAGACTACAAGGGGGAAGGGAAGGAGAGATGAATTAGCTTCCC CTGTAAACCTTAGAACCCAGCTGTTGCCAGGGCAACGGGGCAATACCTGTCTCTTCAG AGGAGATGAAGTTGCCAGGGTAACTACATCCTGTCTTTCTCAAGGACCATCCCAGAAT GTGGCACCCACTAGCCGTTACCATAGCAACTGCCTCTTTGCCCCACTTAATCCCATCCC GTCTGTTAAAAGGGCCCTATAGTTGGAGGTGGGGGAGGTAGGAAGAGCGATGATCACT TGTGGACTAAGTTTGTTCGCATCCCCTTCTCCAACCCCCTCAGTACATCACCCTGGGGG AACAGGGTCCACTTGCTCCTGGGCCCACACAGTCCTGCAGTATTGTGTATATAAGGCCA GGGCAAAGAGGAGCAGGTTTTAAAGTGAAAGGCAGGCAGGTGTTGGGGAGGCAGTTA CCGGGGCAACGGGAACAGGGCGTTTCGGAGGTGGTTGCCATGGGGACCTGGATGCTGA CGAAGGCTCGCGAGGCTGTGAGCAGCCACAGTGCCCTGCTCAGAAGCCCCAAGCTCGT CAGTCAAGCCGGTTCTCCGTTTGCACTCAGGAGCACGGGCAGGCGAGTGGCCCCTAGT TCTGGGGGCAGC (SEQ ID NO: 2 (CAMKII)). In some embodiments, the promotor consists of a nucleic acid and/or amino acid sequence that is about 60%, about 70%, about 80%, about 90%, about 95%, about 99%, about 99.5% or about 100% identity to SEQ ID NO: 2 (CAMKII).

In some embodiments, the promotor comprises a nucleic acid and/or amino acid sequence that is about 60%, about 70%, about 80%, about 90%, about 95%, about 99%, about 99.5% or about 100% identity to CGAAGGATGGGTGCCAGGGAAACTGACTTATAGACGACCCGCTCCGCGAGAGACTGAC AACTCAGTCCTGCGGGCTTTCGGGGCGCCCAGACCAGTCCTTAGCCAGAGGATCTGGG GCTGCACAGCTCCCGGACCCTGGAGGGGGTGGTCCGGGGGGGGAGACTGCGAGCATC AGCCCACGCCGGCCCCCTCCCACCCTGCGGCGGCGAGGTGGGAAGGGCCAGGATGCGA GCTTAGCACCTCCCCTAGAAATGCAGCACTTCGCCCCCCCACCTCCCCTGCCCTCGGCG CTCCCTCTTTCACGCGCTCCCTCCCGCCTCCTTCCCAAGGGCGCCCTTCTTCTGCCCCCA GCTCACGTCTGAATCCCTCGGCGCCCCCTTTCTCTTCTCCTAGCCCCTTCCTCACGTCCC CTGCCTCCGGGTATTTCCTCTCTCCAATCCCCCACCCCCGCACCGCCTGATTCCGAGGG GCGGGAGCGCATTGGGCTGCGCACGGGTGGGGGCGCCGCGCCAGCTTCGCGTAGCTGC TCTGACGCCGCTGCCGCCGCCGCCGCCGCCGCCGCCCTCCGCAGCCCAGCTCGCGCCCC GCGGCAGCTCCGC (SEQ ID NO: 3 (Vgat)). In some embodiments, a promotor consists of a nucleic acid and/or amino acid sequence that is about 60%, about 70%, about 80%, about 90%, about 95%, about 99%, about 99.5% or about 100% identity to SEQ ID NO: 3 (Vgat).

In some embodiments, the promotor comprises a nucleic acid and/or amino acid sequence that is about 60%, about 70%, about 80%, about 90%, about 95%, about 99%, about 99.5% or about 100% identity to a synapsin I promoter. In some embodiments, the promotor comprises a nucleic acid and/or amino acid sequence that is about 60%, about 70%, about 80%, about 90%, about 95%, about 99%, about 99.5% or about 100% identity to GGGGTGGGGGTGCCTACCTGACGACCGACCCCGACCCACTGGACAAGCACCCAACCCC CATTCCCCAAATTGCGCATCCCCTATCAGAGAGGGGGAGGGGAAACAGGATGCGGCGA GGCGCGTGCGCACTGCCAGCTTCAGCACCGCGGACAGTGCCTTCGCCCCCGCCTGGCG GCGCGCGCCACCGCCGCCTCAGCACTGAAGGCGCGCTGACGTCACTCGCCGGTCCCCC GCAAACTCCCCTTCCCGGCCACCTTGGTCGCGTCCGCGCCGCCGCCGGCCCAGCCGGAC CGCACCACGCGAGGCGCGAGATAGGGGGGCACGGGCGCGACCATCTGCGCTGCGGCG CCGGCGACTCAGCGCTGCCTCAGTCTGCGGTGGGCAGCGGAGGAGTCGTGTCGTGCCT GAGAGCGCAG (SEQ ID NO: 4 (hSYN1)). In some embodiments, the promotor consists of a nucleic acid and/or amino acid sequence that is about 60%, about 70%, about 80%, about 90%, about 95%, about 99%, about 99.5% or about 100% identity to SEQ ID NO: 4 (hSYN1).

In some embodiments, the promotor comprises a nucleic acid and/or amino acid sequence that is about 60%, about 70%, about 80%, about 90%, about 95%, about 99%, about 99.5% or about 100% identity to the alpha tubulin 1 promoter. In some embodiments, the promotor consists of a nucleic acid and/or amino acid sequence that is about 60%, about 70%, about 80%, about 90%, about 95%, about 99%, about 99.5% or about 100% identity to the alpha tubulin 1 promoter.

In some embodiments, a promotor comprises a nucleic acid and/or amino acid sequence that is about 60%, about 70%, about 80%, about 90%, about 95%, about 99%, about 99.5% or about 100% identity to GACCCTTAAAGAGAGGAGGGTTAGGAGTCAGGGAGAGGAAAAGGGACCCAAG GGGCCTGGGACCAGCTGAGAAAGACTTAGGAGGCCAGAAGAGTAAGTGAAAAGAATT GGGGTGGCAGGCAGAGGAGTTGGTGGGGGGTGGGGCAGCCATACCTGACACAGAGTG AAGTCGGCTAGGAAAGGACAGGTGTGGGTGCATGGTAGGGGCTGCAGGGGAAAGTTG GTGGTGTATGCAGCTGGACCTAGGAGAGAAGCAGGAGAGGAAGATCCAGCACAAAAA ATCTGAAGCTAAAAACAGGACACAGAGATGGGGGAAGAAAAGAGGGCAGAGTGAGG CAAAAAGAGACTGAAGAGATGAGGGTGGCCGCCAGGCACTTTAGATAGGGGAGAGGC TTTATTTACCTCTGTTTGTTTTTTTTTTTTTTTTTTTTTTTTTTTTGCGAGGTAGTCTTGCTT AGTCTCCAGGCTGGAGTGCAGTGGCACAATCTCAGCTCACTGCAACTTCCACCTCCTGG GTTCAAGCAATTCTCCTGCCTCAGCCTCCCGAGTAGCTGGGACTACAGGCGCATGCAAC CGCGCCTGGCTAATTTTTGTATTTTT (SEQ ID NO: 5 (neuron-specific enolase)). In some embodiments, the promotor consists of a nucleic acid and/or amino acid sequence that is about 60%, about 70%, about 80%, about 90%, about 95%, about 99%, about 99.5% or about 100% identity to SEQ ID NO: 5 (neuron-specific enolase).

In some embodiments, the promotor comprises a nucleic acid and/or amino acid sequence that is about 60%, about 70%, about 80%, about 90%, about 95%, about 99%, about 99.5% or about 100% identity to TCAGCCTCCTGTCTGCCGACCGGGCACCTGCTTGCCCAATGAATATCTTGCTCACTAGC CTGGTGCTTCTGAGGTCAGGACCACGTCTTGCTACTGTCCCCTGCACCCAGTACAGTGT TAGAATGTAGTAAATGCTCAGTATGCTTGTTCAATGAATGAAGGTTGACTGAATCAACC ACAGAGATGATCAGAGTCTCCTTTACAGATGGGGAAACTGAGGCACTGAGAAATGATC TCCCTGGGTGCCATGGCAAGGGGAGGCCGCCACCCGACATGGGCATCGGGAAAGTGG GGCCGAGGCCACTCTTTGACAAAAGTGACTCAGCAGCTGGCGGCTGGCGTGGGCTGTG GAATTTCTAAGCATCCCCTCACATCCTGAGCGAACGGGCGATGGGAGGGAGTAAGCAG AGTGAAAAGAAGAAAAACAAGAAACTTGGGGGAAAAGAAAGAGAGAGGGGGAAAAA AACTGAAATTGAAAACAGACGCACGCGTCCACCCTCCCTGCCCCGCCGCCCCCCCTTCT CCCAGCTGGGCCAATCAGAAGCCACCCTGCAGCCTTTCCCCCAAAGTGGCGGGGGAAG TGGGGGGGCATGGAAGCCC (SEQ ID NO: 6 (a platelet-derived growth factor beta chain promoter)). In some embodiments, the promotor consists of a nucleic acid and/or amino acid sequence that is about 60%, about 70%, about 80%, about 90%, about 95%, about 99%, about 99.5% or about 100% identity to SEQ ID NO: 6 (a platelet-derived growth factor beta chain promoter).

In some embodiments, the promotor comprises a nucleic acid and/or amino acid sequence that is about 60%, about 70%, about 80%, about 90%, about 95%, about 99%, about 99.5% or about 100% identity to GATCTAACATATCCTGGTGTGGAGTAGGGGACGCTGCTCTGACAGAGGCTCGGGGGCC TGAGCTGGCTCTGTGAGCTGGGGAGGAGGCAGACAGCCAGGCCTTGTCTGCAAGCAGA CCTGGCAGCATTGGGCTGGCCGCCCCCCAGGGCCTCCTCTTCATGCCCAGTGAATGACT CACCTTGGCACAGACACAATGTTCGGGGTGGGCACAGTGCCTGCTTCCCGCCGCACCC CAGCCCCCCTCAAATGCCTTCCGAGAAGCCCATTGAGCAGGGGGCTTGCATTGCACCC CAGCCTGACAGCCTGGCATCTTGGGATAAAAGCAGCACAGCCCCCTAGGGGCTGCCCT TGCTGTGTGGCGCCACCGGCGGTGGAGAACAAGGCTCTATTCAGCCTGTGCCCAGGAA AGGGGATCAGGGGATGCCCAGGCATGGACAGTGGGTGGCAGGGGGGGAGAGGAGGGC TGTCTGCTTCCCAGAAGTCCAAGGACACAAATGGGTGAGGGGAGAGCTCTCCCCATAG CTGGGCTGCGGCCCAACCCCACCCCCTCAGGCTATGCCAGGGGGTGTTGCCAGGGGCA CCCGGGCATCGCCAGTCTAGCCCACTCCTTCATAAAGCCCTCGCATCCCAGGAGCGAG CAGAGCCAGAGCAGGTTGGAGAGGAGACGCATCACCTCCGCTGCTCGCGGGG (SEQ ID NO: 7 ((GFAP) promoter)). In some embodiments, the promotor consists of a nucleic acid and/or amino acid sequence that is about 60%, about 70%, about 80%, about 90%, about 95%, about 99%, about 99.5% or about 100% identity to SEQ ID NO: 7 (GFAP) promoter).

In some embodiments, the promotor comprises a nucleic acid and/or amino acid sequence that is about 60%, about 70%, about 80%, about 90%, about 95%, about 99%, about 99.5% or about 100% identity to an cerebellar Purkinje cell-specific L7-6 promoter. In some embodiments, the promotor consists of a nucleic acid and/or amino acid sequence that is about 60%, about 70%, about 80%, about 90%, about 95%, about 99%, about 99.5% or about 100% identity to an cerebellar Purkinje cell-specific L7-6 promoter.

In some embodiments, the promotor comprises a nucleic acid and/or amino acid sequence that is about 60%, about 70%, about 80%, about 90%, about 95%, about 99%, about 99.5% or about 100% identity to GGGGCACCGGCTGGAGTCCGGCGTTGACCGCAGCCGGGACGCGCCCGCCACCTGGGCG GGGAAGGAGTGAGTCCCCTCGGGGCGCAGCGGCGCGCGCTGGCCCGGATCCCAGCTGC GCCGCGCCCTCCCTGCGAGCGGTGAGTACAGGCGCCCACGGGCATCTCCAACCCTAGT TAGGGGACACTTGGAGACAACGCAGGTCGGACGTCCGCCGGCCGTTCTAGGAGTTGGT GTGCGGGGAATGGGGGGTGCTCCCGCTCGCTTAGGGCTTTGGGTCCGGCTAGGGGTTG ACCCCAAGAGGCGCTGGATGTCGGGGCTGAGCTTTGCTTTCAGGGGACTCAAGCTTAC TTGAGGTTCTGACATTGGATTTGCCTCGCGGGCCCTGCGCGTGCTGCGCGGGGATCCGG AGACTGGCGAGGTAACCAGGGAGGGCAAGCACTCACCGGGGCGTCCTCCGCCCACCTC GCCCGCAGAACCATCCAGGGGGACCACGTGCGGCTGTCGCCCGCAACTCTGCCTGTCA AGCGAGGACCGCCCCCAGGGCAGGGGAGGGGACGCGCGGGCGGGGTGGGCTGTGCCC CGCGGGAACCCCGCCGGCCT (SEQ ID NO: 8 (a dopamine receptor D1 (DRD1) promoter)).

In some embodiments, the promotor consists of a nucleic acid and/or amino acid sequence that is about 60%, about 70%, about 80%, about 90%, about 95%, about 99%, about 99.5% or about 100% identity to SEQ ID NO: 8 a dopamine receptor D1 (DRD1) promoter).

In some embodiments, the promotor comprises a nucleic acid and/or amino acid sequence that is about 60%, about 70%, about 80%, about 90%, about 95%, about 99%, about 99.5% or about 100% identity to GTGCGGGATCAGGGGCCTGGTTTTCTTTCTGTGTGTGACTTCTGAATCTGACACAGAAT CTGGCAAACTGAGGACAGTTGGTAACTAATTGCTGCAGAAGAAAGAAAAGAAAGAGA AAGAAAGAAAAAGAAAGGAAGGAAGAAAGAAAGAAAGAAAGGAAAGAAAGAAAGA GAAAGAAAGAAAGAAAGAAAGAAAGAAAGAAAGAAAGAAAGAAAGAAAGAAAGAA AAGAAAAGAAAAGAAAGAAAAGGAGAGAGGAAGAAAAGGGGAGAAGAACGAGGTTC CTCTGCAGCCCAAATGACAGTGGGCAGGGTAGCAGCGGAACACCCTTCACCACTCTAT CTCTTTCTCCCTCTCCCCACTCCCTGCCTCACACACCTCAGTTTGGAGTCAAGGGTGAGC TGGAACCCCTGAGGCCTCCTTTTGCATTTCCATGTCAGGTCAACTCCAAACAGGCTTTT TCCTTTGGATGAGTCCAGCAATAGAACTGACTCATAGATTCCAAGACCTGAAGTCAGA AAACGGATGCGGACCTCTTCCAACACCTCCTCTCCTTGCCTGACTTGCTGGATGGGTGG TGCCTCCCGCAACCCTTGGCTTCTGAGTCCTCAAAGGAGAAGACTGGCGAGCAGACGG TGAGGACCCAGCCTGCAATCACAGCTTATTACTCTGGGTGTGGGTGGGAGCGCAGTGG GCGGTCGAGGGTTGCGTTCCCGCCTCAAAACAAGGGATGGCGGAATCCCCCAACCCCT CCTACCCGTTCAGGCCGGGGATCGCCGAGGAGGTACAGCTCCTTTGGTGGGGGGCGGG GGCGGGGCCTGTCTCAGGGGCGGGGACCGGGGCACCTCCCTCCCGCGCTCCCCGCGCT CGGGCGCCGCAGAGCTGTCCAGCTTC (SEQ ID NO: 9 ((DRD2) promoter)). In some embodiments, the promotor consists of a nucleic acid and/or amino acid sequence that is about 60%, about 70%, about 80%, about 90%, about 95%, about 99%, about 99.5% or about 100% identity to SEQ ID NO: 9 ((DRD2) promoter).

In some embodiments, the promotor comprises a nucleic acid and/or amino acid sequence that is about 60%, about 70%, about 80%, about 90%, about 95%, about 99%, about 99.5% or about 100% identity to AAGGGACTTGTCTGAGAATATGCGGAACATTTCCTTCTACCAGGCACTAGATCCTCGCA CAAAAGTGCTGAGTCCGCTCCCAACCCCAGGCCCGTGGCTTTGAGCAGCAGGTCACTT AACATCTAACGTCTTTATGCTGTTTCCTCATCTGTGCGAGAGCAGCTATGTCTACCTGG CAAGGCTGTAGTGAGAGATCATATCAGCATAGGAATGGGGCTCAGCCCCATGCACAGA GGACAGTTCTTGTTTCATTCTTTTCCTTGCTGTTTCTCTTCCTTTCCTGGCAGAATATGGA GGAAGGAAGCATCGCTGCCATCTACAGTGGTCACGGAAGGCTTCATGGAAGAGGCGA GCCCTGCCTGGGCCTCAATTTTGGGTGCTGGAGGGAAGCAGGGGCCAAGAGTTATTAA TAGTCTTGGCCTGATGGGCCCAGGGAGGCTGAATGTGATACAGACACCCAGCACCACG GTTGGGGAGTACCTGACACCGGAAGGGGAGGGGGCCGGGGCTACGGGGAGTGCCACC TCCCAAAATAGCCAGAGCAGAAGCCTATATAGGTGGCCATCCCACCTCCAGGCTCACT TCCCGACAGGACTTCCCACCAGCCCAGCCTTTCAGTGCAGGCTCCAGCCCTCCACCCCC ACCCGAGGTGAGTGGCAGCTACCGAGGTTGGAGGATAGAGGGATGCAGCAGGATGAG CCAGCTGGAAGGGAGAGCTACATCTCCCCTGTCCGTAGTGACCCGGGGAGGGGGGTGC GGTGGGGGTGCTGGAGGCAGGGCAGCTGTGGAATGTAGGGCTGAGAGCATGCATTCCT GCTTCTCCACCCAGACTCCTGGTGTGGCCTGGGGCAAACTCCCCCCACCCACCGCCCCC CGTGGCTGGGCCTCAGATTCCCCAGCCTTAGAACGAGAGGGCTGGAGTCAGACATCTG TGGACTCTGGTGATGCCATCAGAGAGGACCCAGAGGGCAGAGGAGGACTACCCAGGG CTGATGGGCTGACCTAATGACATTGATGCTCTGGCTGGGAGGGCTCAGTCCCGGAGGG GGTGTCAAGTTGATAGGAGAACTCAGCAGCAGGGAGAGATGGAGGAAGGAGATGGAA GAAGAGTGGGCCCCTGTTGAGGCTGCAGCCTTTGCCACAGGGTAGCTGCTTAGGGGCT CACCAGGAGCCAGTGTGTGGCTGAGATGTATGGGACCTGATAGAGCTGGGTCCCAGGC ACCAGGGAGGATCACCCAGAGAGTCACTGAACTCCACACGAGTGAGTCCAGGGGTGTA CCCTGCTCTGCTATTGCTTTAC (SEQ ID NO: 10 ((parvalbumin (Pvalb) promoter)). In some embodiments, the promotor consists of a nucleic acid and/or amino acid sequence that is about 50%, about 60%, about 70%, about 80%, about 90%, about 95%, about 99%, about 99.5% or about 100% identity to SEQ ID NO: 10 ((parvalbumin (Pvalb) promoter).

In some embodiments, the promotor comprises a nucleic acid and/or amino acid sequence that is about 60%, about 70%, about 80%, about 90%, about 95%, about 99%, about 99.5% or about 100% identity to CACTTTCTAATTACCTAACAAAGTAATTTGGGATTCACCGTGGGGCTGGCGGGATGGG GAACCAACATCCACTTACAGCCAGGGTGGTGTACGCCACACCGCGAGGGACTGGCTGT ATTGGTAGTAGTGGCGATGGGGTGGGGGCTCTTGTGCACTTTTTTGTACCTAGCACCCC AGAAGACAGAAGGAGCAGCGGCAGATCTTGCCTCCACTAGCCCTGCCCCCTAGGCCTG GTGGCGCGCGCTTTGGCCACTGGAGGTGAGGAAGATCCTGGGTGGCAACGAGAGACGT CTACACATAGATAAGGGAGCCTACCACAGGCCTGGGTTTGCTGCGGGTTTCGGTGTCCC CCAGGGACTCCTAAACTCTGGAAGTCGCGCTAGACCTCAGGAGGCCGGAAAATGACCA CCTGGCGTCGGTGGCTCCAGCAGATCTGAGCCCCTAGGCCCCCTGGGCTCCACGCTGA GCCGCGCTCTCGGGGCGAGTGAGCCCGGGGCTCTCCGCGCATGGGGAGTGGAGAAGG GAGGGGGAGGAGGAGGAAGGCTGGGAGGGAAAGAGGATGTGTGTGTTGGGGGAGGG GGCGGTGCAGCCGCCTTTCCT (SEQ ID NO: 11 (Dlx) promoter)). In some embodiments, the promotor consists of a nucleic acid and/or amino acid sequence that is about 60%, about 70%, about 80%, about 90%, about 95%, about 99%, about 99.5% or about 100% identity to SEQ ID NO: 11 (Dlx) promoter).

In some embodiments, the promotor comprises a nucleic acid and/or amino acid sequence that is about 60%, about 70%, about 80%, about 90%, about 95%, about 99%, about 99.5% or about 100% identity TOGGAGTTCCGCGTTACATAACTTACGGTAAATGGCCCGCCTGGCTGACCGCCCAACG ACCCCCGCCCATTGACGTCAATAATGACGTATGTTCCCATAGTAACGCCAATAGGGACT TTCCATTGACGTCAATGGGTGGAGTATTTACGGTAAACTGCCCACTTGGCAGTACATCA AGTGTATCATATGCCAAGTACGCCCCCTATTGACGTCAATGACGGTAAATGGCCCGCCT GGCATTATGCCCAGTACATGACCTTATGGGACTTTCCTACTTGGCAGTACATCTACGTA TTAGTCATCGCTATTACCATGGTGATGCGGTTTTGGCAGTACATCAATGGGCGTGGATA GCGGTTTGACTCACGGGGATTTCCAAGTCTCCACCCCATTGACGTCAATGGGAGTTTGT TTTGGCACCAAAATCAACGGGACTTTCCAAAATGTCGTAACAACTCCGCCCCATTGACG CAAATGGGCGGTAGGCGTGTACGGTGGGAGGTCTATATAAGCAGAGCTCGTTTAGTGA ACCGTCAGATCGCCTGGAGACGCCATCCACGCTGTTTTGACCTCCATAGAAGACACCG (SEQ ID NO: 12 (CMV)). In some embodiments, the promotor consists of a nucleic acid and/or amino acid sequence that is about 60%, about 70%, about 80%, about 90%, about 95%, about 99%, about 99.5% or about 100% identity to SEQ ID NO: 12 (CMV).

In some embodiments, the promoter is operatively coupled to a gene of interest. In some embodiments, the promoter is operatively coupled to an open reading frame of a gene of interest. In some embodiments, the gene of interest comprises a DREADD, such as a DREADD described below.

### Gene of Interest

### Designer Receptors Exclusively Activated by Designer Drugs (DREADDs)

In some aspects, a composition and/or gene therapy construct of the present disclosure comprises a designer receptor exclusively activated by designer drugs (DREADD) as the gene of interest. A DREADD, also known as a receptor, activated solely by a synthetic ligand, can be a class of artificially engineered protein receptors which can be selectively activated by certain ligands. In some embodiments, the DREADD can be a Gi-DREADD, a Gq-DREADD, a Gs-DREADD, an Arrestin-DREADD, an Axonally-targeted silencing DREADD, or a KORD-DREADD. In some embodiments, the DREADD can be inhibitory and/or excitatory or a combination of inhibitory and excitatory. In some embodiments, DREADDs comprise modified versions of natural excitatory or inhibitory GPCRs. The modified versions can be engineered to respond to synthetic molecules rather than endogenous ligands.

### Excitatory DREADDS

In some embodiments, the excitatory DREADD comprises a Gq-DREADD. In some embodiments, the Gq-DREADD comprises hM1Dq, hM3Dq, and/or hM5Dq.

In some embodiments, the excitatory DREADD can be introduced to the neuron via administration of a pharmaceutical composition comprising the excitatory DREADD to a subject. In some embodiments, an excitatory DREADD comprises a nucleic acid and/or amino acid sequence that is about 60%, about 70%, about 80%, about 90%, about 95%, about 99%, about 99.5% or about 100% identity to SEQ ID NO: 1 (hM3Dq). In some embodiments, an excitatory DREADD promotor consists of a nucleic acid and/or amino acid sequence that is about 60%, about 70%, about 80%, about 90%, about 95%, about 99%, about 99.5% or about 100% identity to SEQ ID NO: 1 (hM3Dq).

In some embodiments, activation of an excitatory DREADD increases neuronal firing by about 10%, about 20%, about 30%, about 40%, about 50%, about 60%, about 70%, about 80%, about 90%, or about 100%, compared to a neuron that does not express the excitatory DREADD. In some embodiments, the excitatory DREADD can increase neuronal firing from about 10% to about 100%, about 20% to about 90%, about 30% to about 80%, about 40% to about 70%, or about 50% to about 60%, compared to a neuron that does not express the excitatory DREADD.

In some embodiments, activation of an excitatory DREADD increases activation or release of phospholipase C by about 10%, about 20%, about 30%, about 40%, about 50%, about 60%, about 70%, about 80%, about 90%, or about 100%, compared to a neuron that does not express the excitatory DREADD. In some embodiments, the excitatory DREADD can increase activation or release of phospholipase C from about 10% to about 100%, about 20% to about 90%, about 30% to about 80%, about 40% to about 70%, or about 50% to about 60% compared to a neuron that does not express the excitatory DREADD.

In some embodiments, activation of an excitatory DREADD increases activation or release of Inositol trisphosphate (IP3) by about 10%, about 20%, about 30%, about 40%, about 50%, about 60%, about 70%, about 80%, about 90%, or about 100%, compared to a neuron that does not express the excitatory DREADD. In some embodiments, the excitatory DREADD can increase activation or release of IP3 from about 10% to about 100%, about 20% to about 90%, about 30% to about 80%, about 40% to about 70%, or about 50% to about 60% compared to a neuron that does not express the excitatory DREADD.

In some embodiments, activation of an excitatory DREADD increases activation or release of Diacylglycerol (DAG) by about 10%, about 20%, about 30%, about 40%, about 50%, about 60%, about 70%, about 80%, about 90%, or about 100%, compared to a neuron that does not express the excitatory DREADD. An excitatory DREADD can increase activation or release of DAG from about 10% to about 100%, about 20% to about 90%, about 30% to about 80%, about 40% to about 70%, or about 50% to about 60% compared to a neuron that does not express the excitatory DREADD.

In some embodiments, activation of an excitatory DREADD increases activation or release of intracellular calcium by about 10%, about 20%, about 30%, about 40%, about 50%, about 60%, about 70%, about 80%, about 90%, or about 100%, compared to a neuron that does not express the excitatory DREADD. In some embodiments, the excitatory DREADD can increase activation or release of intracellular calcium from about 10% to about 100%, about 20% to about 90%, about 30% to about 80%, about 40% to about 70%, or about 50% to about 60% compared to a neuron that does not express the excitatory DREADD.

In some embodiments, an excitatory DREADD can be administered to a neuron. In some embodiments, the neuron comprises a glutamatergic neuron, a GABAergic neuron, a dopaminergic neuron, or a serotonin neuron. In some embodiments, the neuron comprises a neuron of the inferior colliculus. In some embodiments, the neuron comprises a neuron of the caudate nucleus. In some embodiments, the neuron comprises a neuron of the left caudate nucleus. In some embodiments, the neuron comprises a neuron of the right caudate nucleus.

In some embodiments, a symptom of a neurological disease and/or condition in a subject can be reduced when an excitatory DREADD is introduced to a neuron of a subject. In some embodiments, the symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, a symptom of a neurological disease and/or condition in a subject can be reduced when an excitatory DREADD is introduced to a glutamatergic neuron of a subject. The symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, a symptom of a neurological disease and/or condition in a subject can be reduced when an excitatory DREADD is introduced to a GABAergic neuron of a subject. The symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, a symptom of a neurological disease and/or condition in a subject can be reduced when an excitatory DREADD is administered to a dopaminergic neuron of a subject. The symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, a symptom of a neurological disease and/or condition in a subject is reduced when an excitatory DREADD is administered to a serotonin neuron of a subject. The symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, a symptom of a neurological disease and/or condition in a subject can be reduced when an excitatory DREADD is introduced to a neuron of the caudate nucleus of a subject. The symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, a symptom of a neurological disease and/or condition in a subject can be reduced when an excitatory DREADD is introduced to a neuron of the left caudate nucleus of a subject. The symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, a symptom of a neurological disease and/or condition in a subject can be reduced when an excitatory DREADD is introduced to a neuron of the right caudate nucleus of a subject. The symptom of the subject can be reduced by about 10%, about 20%, about 30%, about 40%, about 50%, about 60%, about 70%, about 80%, about 90%, or about 100% compared to a subject that does not comprise the excitatory DREADD. In some embodiments, the symptom comprises vertigo, dizziness, hearing impairment, buzzing, clicking, humming, hissing, roaring, or ringing, or a combination of these symptoms. In some embodiments, the vertigo is reduced by about 10%, about 20%, about 30%, about 40%, about 50%, about 60%, about 70%, about 80%, about 90%, or about 100% compared to a subject that does not comprise the excitatory DREADD. In some embodiments, the dizziness is reduced by about 10%, about 20%, about 30%, about 40%, about 50%, about 60%, about 70%, about 80%, about 90%, or about 100% compared to a subject that does not comprise the excitatory DREADD. In some embodiments, the hearing impairment is reduced by about 10%, about 20%, about 30%, about 40%, about 50%, about 60%, about 70%, about 80%, about 90%, or about 100% compared to a subject that does not comprise the excitatory DREADD. In some embodiments, the buzzing is reduced by about 10%, about 20%, about 30%, about 40%, about 50%, about 60%, about 70%, about 80%, about 90%, or about 100% compared to a subject that does not comprise the excitatory DREADD. In some embodiments, the clicking is reduced by about 10%, about 20%, about 30%, about 40%, about 50%, about 60%, about 70%, about 80%, about 90%, or about 100% compared to a subject that does not comprise the excitatory DREADD. In some embodiments, the humming is reduced by about 10%, about 20%, about 30%, about 40%, about 50%, about 60%, about 70%, about 80%, about 90%, or about 100% compared to a subject that does not comprise the excitatory DREADD. In some embodiments, the hissing is reduced by about 10%, about 20%, about 30%, about 40%, about 50%, about 60%, about 70%, about 80%, about 90%, or about 100% compared to a subject that does not comprise the excitatory DREADD. In some embodiments, the roaring is reduced by about 10%, about 20%, about 30%, about 40%, about 50%, about 60%, about 70%, about 80%, about 90%, or about 100% compared to a subject that does not comprise the excitatory DREADD. In some embodiments, the ringing is reduced by about 10%, about 20%, about 30%, about 40%, about 50%, about 60%, about 70%, about 80%, about 90%, or about 100% compared to a subject that does not comprise the excitatory DREADD.

In some embodiments, about 5%, about 10%, about 20%, about 30%, about 40%, about 50%, about 60%, about 70%, about 80%, about 90%, or about 100% of neurons within the inferior colliculus comprise the excitatory DREADD.

In some embodiments, about 5%, about 10%, about 20%, about 30%, about 40%, about 50%, about 60%, about 70%, about 80%, about 90%, or about 100% of neurons within the Caudate Nucleus comprise the excitatory DREADD.

In some embodiments, about 5%, about 10%, about 20%, about 30%, about 40%, about 50%, about 60%, about 70%, about 80%, about 90%, or about 100% of neurons within the left Caudate Nucleus comprise the excitatory DREADD.

In some embodiments, about 5%, about 10%, about 20%, about 30%, about 40%, about 50%, about 60%, about 70%, about 80%, about 90%, or about 100% of neurons within the right caudate nucleus comprise the excitatory DREADD.

In some embodiments, about 5%, by about 10%, about 20%, about 30%, about 40%, about 50%, about 60%, about 70%, about 80%, about 90%, or about 100% of the glutamatergic neurons within the caudate nucleus comprise the excitatory DREADD.

In some embodiments, about 5%, about 10%, about 20%, about 30%, about 40%, about 50%, about 60%, about 70%, about 80%, about 90%, or about 100% of the GABAergic neurons within the caudate nucleus comprise the excitatory DREADD.

In some embodiments, about 5%, about 10%, about 20%, about 30%, about 40%, about 50%, about 60%, about 70%, about 80%, about 90%, or about 100% of the dopaminergic neurons within the caudate nucleus comprise the excitatory DREADD.

In some embodiments, about 5%, about 10%, about 20%, about 30%, about 40%, about 50%, about 60%, about 70%, about 80%, about 90%, or about 100% of the serotonin neurons within the caudate nucleus comprise the excitatory DREADD.

In some embodiments, about 5%, about 10%, about 20%, about 30%, about 40%, about 50%, about 60%, about 70%, about 80%, about 90%, or about 100% of the glutamatergic neurons within the left caudate nucleus comprise the excitatory DREADD.

In some embodiments, about 5%, about 10%, about 20%, about 30%, about 40%, about 50%, about 60%, about 70%, about 80%, about 90%, or about 100% of the GABAergic neurons within the left caudate nucleus comprise the excitatory DREADD.

In some embodiments, about 5%, about 10%, about 20%, about 30%, about 40%, about 50%, about 60%, about 70%, about 80%, about 90%, or about 100% of the dopaminergic neurons within the left caudate nucleus comprise the excitatory DREADD.

In some embodiments, about 5%, about 10%, about 20%, about 30%, about 40%, about 50%, about 60%, about 70%, about 80%, about 90%, or about 100% of the serotonin neurons within the left caudate nucleus comprise the excitatory DREADD.

In some embodiments, about 5%, about 10%, about 20%, about 30%, about 40%, about 50%, about 60%, about 70%, about 80%, about 90%, or about 100% of the glutamatergic neurons within the right caudate nucleus comprise the excitatory DREADD.

In some embodiments, about 5%, about 10%, about 20%, about 30%, about 40%, about 50%, about 60%, about 70%, about 80%, about 90%, or about 100% of the GABAergic neurons within the right caudate nucleus comprise the excitatory DREADD.

In some embodiments, about 5%, about 10%, about 20%, about 30%, about 40%, about 50%, about 60%, about 70%, about 80%, about 90%, or about 100% of the dopaminergic neurons within the right caudate nucleus comprise the excitatory DREADD.

In some embodiments, about 5%, about 10%, about 20%, about 30%, about 40%, about 50%, about 60%, about 70%, about 80%, about 90%, or about 100% of the serotonin neurons within the right caudate nucleus comprise the excitatory DREADD.

In some embodiments, about 5%, about 10%, about 20%, about 30%, about 40%, about 50%, about 60%, about 70%, about 80%, about 90%, or about 100% of the glutamatergic neurons within the inferior colliculus comprise the excitatory DREADD.

In some embodiments, about 5%, about 10%, about 20%, about 30%, about 40%, about 50%, about 60%, about 70%, about 80%, about 90%, or about 100% of the GABAergic neurons within the inferior colliculus comprise the excitatory DREADD.

In some embodiments, about 5%, about 10%, about 20%, about 30%, about 40%, about 50%, about 60%, about 70%, about 80%, about 90%, or about 100% of the dopaminergic neurons within the inferior colliculus comprise the excitatory DREADD.

In some embodiments, about 5%, about 10%, about 20%, about 30%, about 40%, about 50%, about 60%, about 70%, about 80%, about 90%, or about 100% of the serotonin neurons within the inferior colliculus comprise the excitatory DREADD.

### Inhibitory DREADDS

In some embodiments, the DREADD comprises a Gi-DREADD. In some embodiments, the Gi-DREADD comprises an hM2Di, hM4Di, GsD, or KORD DREADD.

In some embodiments, the dread comprises an inhibitory DREADD. In some embodiments, the inhibitory DREADD comprises a nucleic acid and/or amino acid sequence that is about 60%, about 70%, about 80%, about 90%, about 95%, about 99%, about 99.5% or about 100% identity to ATGGCCAACTTCACACCTGTCAATGGCAGCTCGGGCAATCAGTCCGTGCGCCTGGTCAC GTCATCATCCCACAATCGCTATGAGACGGTGGAAATGGTCTTCATTGCCACAGTGACA GGCTCCCTGAGCCTGGTGACTGTCGTGGGCAACATCCTGGTGATGCTGTCCATCAAGGT CAACAGGCAGCTGCAGACAGTCAACAACTACTTCCTCTTCAGCCTGGCGTGTGCTGATC TCATCATAGGCGCCTTCTCCATGAACCTCTACACCGTGTACATCATCAAGGGCTACTGG CCCCTGGGCGCCGTGGTCTGCGACCTGTGGCTGGCCCTGGACTGCGTGGTGAGCAACG CCTCCGTCATGAACCTTCTCATCATCAGCTTTGACCGCTACTTCTGCGTCACCAAGCCTC TCACCTACCCTGCCCGGCGCACCACCAAGATGGCAGGCCTCATGATTGCTGCTGCCTGG GTACTGTCCTTCGTGCTCTGGGCGCCTGCCATCTTGTTCTGGCAGTTTGTGGTGGGTAA GCGGACGGTGCCCGACAACCAGTGCTTCATCCAGTTCCTGTCCAACCCAGCAGTGACCT TTGGCACAGCCATTGCTGGCTTCTACCTGCCTGTGGTCATCATGACGGTGCTGTACATC CACATCTCCCTGGCCAGTCGCAGCCGAGTCCACAAGCACCGGCCCGAGGGCCCGAAGG AGAAGAAAGCCAAGACGCTGGCCTTCCTCAAGAGCCCACTAATGAAGCAGAGCGTCA AGAAGCCCCCGCCCGGGGAGGCCGCCCGGGAGGAGCTGCGCAATGGCAAGCTGGAGG AGGCCCCCCCGCCAGCGCTGCCACCGCCACCGCGCCCCGTGGCTGATAAGGACACTTC CAATGAGTCCAGCTCAGGCAGTGCCACCCAGAACACCAAGGAACGCCCAGCCACAGA GCTGTCCACCACAGAGGCCACCACGCCCGCCATGCCCGCCCCTCCCCTGCAGCCGCGG GCCCTCAACCCAGCCTCCAGATGGTCCAAGATCCAGATTGTGACGAAGCAGACAGGCA ATGAGTGTGTGACAGCCATTGAGATTGTGCCTGCCACGCCGGCTGGCATGCGCCCTGC GGCCAACGTGGCCCGCAAGTTCGCCAGCATCGCTCGCAACCAGGTGCGCAAGAAGCGG CAGATGGCGGCCCGGGAGCGCAAAGTGACACGAACGATCTTTGCCATTCTGCTGGCCT TCATCCTCACCTGGACGCCCTACAACGTCATGGTCCTGGTGAACACCTTCTGCCAGAGC TGCATCCCTGACACGGTGTGGTCCATTGGCTACTGGCTCTGCTACGTCAACAGCACCAT CAACCCTGCCTGCTATGCTCTGTGCAACGCCACCTTTAAAAAGACCTTCCGGCACCTGC TGCTGTGCCAGTATCGGAACATCGGCACTGCCAGGCG (SEQ ID NO: 13 (hM4Di)). In some embodiments, an excitatory DREADD promotor consists of a nucleic acid and/or amino acid sequence that is about 60%, about 70%, about 80%, about 90%, about 95%, about 99%, about 99.5% or about 100% identity to SEQ ID NO: 13 (hM4Di).

In some embodiments, an inhibitory DREADD can be introduced to a neuron. In some embodiments, the inhibitory DREADD can be introduced to the neuron via administration of a pharmaceutical composition comprising the inhibitory DREADD to a subject. In some embodiments, the neuron is a glutamatergic neuron, a GABAergic neuron, a dopaminergic neuron, or a serotonin neuron. In some embodiments, a neuron expressing an inhibitory DREADD can be activated by a small molecule actuator.

In some embodiments, activation of an inhibitory DREADD decreases neuronal firing by about 10%, about 20%, about 30%, about 40%, about 50%, about 60%, about 70%, about 80%, about 90%, or about 100%, compared to a neuron that does not express the inhibitory DREADD. In some embodiments, an inhibitory DREADD can decrease neuronal firing from about 10% to about 100%, about 20% to about 90%, about 30% to about 80%, about 40% to about 70%, or about 50% to about 60% compared to a neuron that does not express the inhibitory DREADD.

In some embodiments, activation of an inhibitory DREADD decreases activation or release of cyclic adenosine monophosphate (cAMP) by about 10%, about 20%, about 30%, about 40%, about 50%, about 60%, about 70%, about 80%, about 90%, or about 100%, compared to a neuron that does not express the inhibitory DREADD. In some embodiments, the inhibitory DREADD can decrease activation or release of cAMP from about 10% to about 100%, about 20% to about 90%, about 30% to about 80%, about 40% to about 70%, or about 50% to about 60% compared to a neuron that does not express the inhibitory DREADD.

In some embodiments, activation of an inhibitory DREADD decreases activation or release of intracellular calcium by about 10%, about 20%, about 30%, about 40%, about 50%, about 60%, about 70%, about 80%, about 90%, or about 100%, compared to a neuron that does not express the inhibitory DREADD. In some embodiments, the inhibitory DREADD can decrease activation or release of intracellular calcium from about 10% to about 100%, about 20% to about 90%, about 30% to about 80%, about 40% to about 70%, or about 50% to about 60% compared to a neuron that does not express the inhibitory DREADD.

In some embodiments, activation of an inhibitory DREADD increases activation or release of potassium by about 10%, about 20%, about 30%, about 40%, about 50%, about 60%, about 70%, about 80%, about 90%, or about 100%, compared to a neuron that does not express the inhibitory DREADD. In some embodiments, the inhibitory DREADD can increase activation or release of potassium from about 10% to about 100%, about 20% to about 90%, about 30% to about 80%, about 40% to about 70%, or about 50% to about 60% compared to a neuron that does not express the inhibitory DREADD.

In some embodiments, a symptom of a neurological disease and/or condition in a subject can be reduced when an inhibitory DREADD is introduced to a neuron of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD.

In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, a symptom of a neurological disease and/or condition in a subject can be reduced when an inhibitory DREADD is introduced to a glutamatergic neuron of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD. In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, a symptom of a neurological disease and/or condition in a subject can be reduced when an inhibitory DREADD is introduced to a GABAergic neuron of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD. In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, a symptom of a neurological disease and/or condition in a subject can be reduced when an inhibitory DREADD is introduced to a dopaminergic neuron of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD. In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, a symptom of a neurological disease and/or condition in a subject can be reduced when an inhibitory DREADD is introduced to a serotonin neuron of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD. In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, a symptom of a neurological disease and/or condition in a subject can be reduced when an inhibitory DREADD is introduce to a neuron of the caudate nucleus of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD. In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, a symptom of a neurological disease and/or condition in a subject can be reduced when an inhibitory DREADD is introduced to a neuron of the left caudate nucleus of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD. In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, a symptom of a neurological disease and/or condition in a subject is reduced when an inhibitory DREADD is administered to a neuron of the right caudate nucleus of a subject. The symptom of the subject can be reduced by about 10%, about 20%, about 30%, about 40%, about 50%, about 60%, about 70%, about 80%, about 90%, or about 100% compared to a subject that does not comprise the inhibitory DREADD. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the inhibitory DREADD. In some embodiments, the dizziness is reduced by about 10%, about 20%, about 30%, about 40%, about 50%, about 60%, about 70%, about 80%, about 90%, or about 100% compared to a subject that does not comprise the inhibitory DREADD. In some embodiments, the hearing impairment is reduced by about 10%, about 20%, about 30%, about 40%, about 50%, about 60%, about 70%, about 80%, about 90%, or about 100% compared to a subject that does not comprise the inhibitory DREADD. In some embodiments, the buzzing is reduced by about 10%, about 20%, about 30%, about 40%, about 50%, about 60%, about 70%, about 80%, about 90%, or about 100% compared to a subject that does not comprise the inhibitory DREADD. In some embodiments, the clicking is reduced by about 10%, about 20%, about 30%, about 40%, about 50%, about 60%, about 70%, about 80%, about 90%, or about 100% compared to a subject that does not comprise the inhibitory DREADD. In some embodiments, the humming is reduced by about 10%, about 20%, about 30%, about 40%, about 50%, about 60%, about 70%, about 80%, about 90%, or about 100% compared to a subject that does not comprise the inhibitory DREADD. In some embodiments, the hissing is reduced by about 10%, about 20%, about 30%, about 40%, about 50%, about 60%, about 70%, about 80%, about 90%, or about 100% compared to a subject that does not comprise the inhibitory DREADD. In some embodiments, the roaring is reduced by about 10%, about 20%, about 30%, about 40%, about 50%, about 60%, about 70%, about 80%, about 90%, or about 100% compared to a subject that does not comprise the inhibitory DREADD. In some embodiments, the ringing is reduced by about 10%, about 20%, about 30%, about 40%, about 50%, about 60%, about 70%, about 80%, about 90%, or about 100% compared to a subject that does not comprise the inhibitory DREADD.

In some embodiments, about 5%, about 10%, about 20%, about 30%, about 40%, about 50%, about 60%, about 70%, about 80%, about 90%, or about 100% of neurons within the inferior colliculus comprise the inhibitory DREADD.

In some embodiments, about 5%, about 10%, about 20%, about 30%, about 40%, about 50%, about 60%, about 70%, about 80%, about 90%, or about 100% of neurons within the Caudate Nucleus comprise the inhibitory DREADD.

In some embodiments, about 5%, about 10%, about 20%, about 30%, about 40%, about 50%, about 60%, about 70%, about 80%, about 90%, or about 100% of neurons within the left Caudate Nucleus comprise the inhibitory DREADD.

In some embodiments, about 5%, about 10%, about 20%, about 30%, about 40%, about 50%, about 60%, about 70%, about 80%, about 90%, or about 100% of neurons within the right caudate nucleus comprise the inhibitory DREADD.

In some embodiments, about 5%, about 10%, about 20%, about 30%, about 40%, about 50%, about 60%, about 70%, about 80%, about 90%, or about 100% of neurons within the right caudate nucleus comprise the inhibitory DREADD.

In some embodiments, about 5%, about 10%, about 20%, about 30%, about 40%, about 50%, about 60%, about 70%, about 80%, about 90%, or about 100% of the glutamatergic neurons within the caudate nucleus comprise the inhibitory DREADD.

In some embodiments, about 5%, about 10%, about 20%, about 30%, about 40%, about 50%, about 60%, about 70%, about 80%, about 90%, or about 100% of the GABAergic neurons within the caudate nucleus comprise the inhibitory DREADD.

In some embodiments, about 5%, about 10%, about 20%, about 30%, about 40%, about 50%, about 60%, about 70%, about 80%, about 90%, or about 100% of the dopaminergic neurons within the caudate nucleus comprise the inhibitory DREADD.

In some embodiments, about 5%, about 10%, about 20%, about 30%, about 40%, about 50%, about 60%, about 70%, about 80%, about 90%, or about 100% of the serotonin neurons within the caudate nucleus comprise the inhibitory DREADD.

In some embodiments, about 5%, about 10%, about 20%, about 30%, about 40%, about 50%, about 60%, about 70%, about 80%, about 90%, or about 100% of the glutamatergic neurons within the left caudate nucleus comprise the inhibitory DREADD.

In some embodiments, about 5%, about 10%, about 20%, about 30%, about 40%, about 50%, about 60%, about 70%, about 80%, about 90%, or about 100% of the GABAergic neurons within the left caudate nucleus comprise the inhibitory DREADD.

In some embodiments, about 5%, about 10%, about 20%, about 30%, about 40%, about 50%, about 60%, about 70%, about 80%, about 90%, or about 100% of the dopaminergic neurons within the left caudate nucleus comprise the inhibitory DREADD.

In some embodiments, about 5%, about 10%, about 20%, about 30%, about 40%, about 50%, about 60%, about 70%, about 80%, about 90%, or about 100% of the serotonin neurons within the left caudate nucleus comprise the inhibitory DREADD.

In some embodiments, about 5%, about 10%, about 20%, about 30%, about 40%, about 50%, about 60%, about 70%, about 80%, about 90%, or about 100% of the glutamatergic neurons within the right caudate nucleus comprise the inhibitory DREADD.

In some embodiments, about 5%, about 10%, about 20%, about 30%, about 40%, about 50%, about 60%, about 70%, about 80%, about 90%, or about 100% of the GABAergic neurons within the right caudate nucleus comprise the inhibitory DREADD.

In some embodiments, about 5%, about 10%, about 20%, about 30%, about 40%, about 50%, about 60%, about 70%, about 80%, about 90%, or about 100% of the dopaminergic neurons within the right caudate nucleus comprise the inhibitory DREADD.

In some embodiments, about 5%, about 10%, about 20%, about 30%, about 40%, about 50%, about 60%, about 70%, about 80%, about 90%, or about 100% of the serotonin neurons within the right caudate nucleus comprise the inhibitory DREADD.

In some embodiments, about 5%, about 10%, about 20%, about 30%, about 40%, about 50%, about 60%, about 70%, about 80%, about 90%, or about 100% of the glutamatergic neurons within the inferior colliculus comprise the inhibitory DREADD.

In some embodiments, about 5%, about 10%, about 20%, about 30%, about 40%, about 50%, about 60%, about 70%, about 80%, about 90%, or about 100% of the GABAergic neurons within the inferior colliculus comprise the inhibitory DREADD.

In some embodiments, about 5%, about 10%, about 20%, about 30%, about 40%, about 50%, about 60%, about 70%, about 80%, about 90%, or about 100% of the dopaminergic neurons within the inferior colliculus comprise the inhibitory DREADD.

In some embodiments, about 5%, about 10%, about 20%, about 30%, about 40%, about 50%, about 60%, about 70%, about 80%, about 90%, or about 100% of the serotonin neurons within the inferior colliculus comprise the inhibitory DREADD.

### Small Molecule Actuators

In some embodiments, the compositions and methods of the disclosure comprise a small molecule actuator. The small molecule actuator can comprise a ligand. The small molecule actuator can comprise an atypical antipsychotic.

In some embodiments, a method of treating a subject with a composition or method of the disclosure can comprise administering a small molecule actuator to the subject to activate a genetically engineered neuron comprising a DREADD. The genetically engineered neuron can comprise a modified GPCR. The small molecule activator can activate a modified GPCR in a neuron of a subject, for example a Gi-DREADD, a Gq-DREADD, a Gs-DREADD, an Arrestin-DREADD, an Axonally-targeted silencing DREADD, or a KORD-DREADD.

In some embodiments, the small molecule actuator comprises Clozapine N-oxide, Clozapine N-oxide dihydrochloride, Deschloroclozapine, DREADD agonist 21, DREADD agonist 21 dihydrochloride, JHU 37152, JHU 37160, Olanzapine, Perlapine, Salvinorin B, and/or Sorbic acid.

In some embodiments, the small molecule actuator is introduced to a cell at a time that is subsequent to a time that the DREADD is introduced to the cell.

In some embodiments, the small molecule actuator is introduced to a cell and/or subject at least 1 day, at least 2 days, at least 3 days, at least 4 days, at least 5 days, at least 6 days, at least 1 week, at least 2 weeks, at least 3 weeks, at least 1 month, or at least more than 1 month following the administration of the DREADD to the cell and/or subject.

In some embodiments, the small molecule actuator is introduced to a cell and/or subject at least 1 time, 2 times, 3 times, 4 times, 5 times, 6 times, 7 times, or more than 7 times per day.

In some embodiments, the small molecule actuator is introduced by injection, and/or intraperitoneal injection, or by any method suitable and known to those skilled in the art.

In some embodiments, the small molecule actuator can have a at least about 10 minute, about 20 minute, about 30 minute, about 40 minute, about 50 minute, about 60 minute, about 70 minute, about 80 minute, about 90 minute, or about 100 minutes residence time in a subject following injection.

In some embodiments, a small molecule actuator can be introduced to a subject in doses comprising from about 0.5 mg/kg, about 1/0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10.0 mg/kg, about 10.5 mg/kg, about 11.0 mg/kg, about 11.5 mg/kg, about 12.0 mg/kg, about 12.5 mg/kg, about 13.0 mg/kg, about 13.5 mg/kg, about 14.0 mg/kg, about 14.5 mg/kg, about 15.0 mg/kg, about 15.5 mg/kg, about 16.0 mg/kg, about 16.5 mg/kg, about 17.0 mg/kg, about 17.5 mg/kg, about 18.0 mg/kg, about 18.5 mg/kg, about 19.0 mg/kg, about 19.5 mg/kg, or about 20.0 mg/kg.

In some embodiments, the small molecule actuator is administered to a subject once every 2 hours, once every three hours, or once every four hours, for a period of at least one day, at least two days, at least three days, at least four days, at least five days, at least six days, at least one week, at least two weeks, at least three weeks, at least one month, or at least until one or more symptoms of a neurological disease or condition have been treated or resolved.

In some embodiments, comprising the methods and compositions described herein, the small molecule actuator cancan be administrated to a subject through various administration routes including oral ingestion, intravenous, intraperitoneal, or subcutaneous injections, inhalation, intranasal application and others as will be recognized by a person skilled in the art. In preferred embodiments, the small molecule actuator cancan be introduced by intravenous administration. The small molecule actuators cancan be in a form of an aqueous solution, solid powder, tablets, aerosols or other forms as will be understood by a person skilled in the art.

In some embodiments, a method of treating a subject with a disease or condition comprises activating a neuron comprising a protein encoded by a heterologous nucleic acid described herein by administering a small molecule activator of the disclosure, thereby activating the protein, thereby treating the disease or condition.

In some embodiments, administration of a small molecule actuator cancan result in activation of a protein encoded by a heterologous nucleic acid. The protein can be expressed in a neuron.

In some embodiments, activation of a neuron comprises increasing neuronal firing by about 10%, about 20%, about 30%, about 40%, about 50%, about 60%, about 70%, about 80%, about 90%, or about 100%, compared to a neuron that is not activated by the small molecule actuator. In some embodiments, the small molecule actuator can increase neuronal firing from about 10% to about 100%, about 20% to about 90%, about 30% to about 80%, about 40% to about 70%, or about 50% to about 60% compared to a neuron that is not activated by the small molecule actuator.

In some embodiments, activation of a neuron comprises increasing activation or release of phospholipase C by about 10%, about 20%, about 30%, about 40%, about 50%, about 60%, about 70%, about 80%, about 90%, or about 100%, compared to a neuron that is not activated. In some embodiments, the small molecule actuator can increase activation or release of phospholipase C from about 10% to about 100%, about 20% to about 90%, about 30% to about 80%, about 40% to about 70%, or about 50% to about 60% compared to a neuron that has not been activated by a small molecule actuator.

In some embodiments, activation of a neuron comprises increasing activation or release of Inositol trisphosphate (IP3) by about 10%, about 20%, about 30%, about 40%, about 50%, about 60%, about 70%, about 80%, about 90%, or about 100%, compared to a neuron that has not been activated. A small molecule actuator can increase activation or release of IP3 from about 10% to about 100%, about 20% to about 90%, about 30% to about 80%, about 40% to about 70%, or about 50% to about 60% compared to a neuron that has not been activated by a small molecule actuator.

In some embodiments, activation of a neuron comprises increasing activation or release of Diacylglycerol (DAG) by about 10%, about 20%, about 30%, about 40%, about 50%, about 60%, about 70%, about 80%, about 90%, or about 100%, compared to a neuron that has not been activated by a small molecule actuator. A small molecule actuator can increase activation or release of DAG from about 10% to about 100%, about 20% to about 90%, about 30% to about 80%, about 40% to about 70%, or about 50% to about 60% compared to a neuron that has not been activated by a small molecule actuator.

In some embodiments, activation of an excitatory DREADD increases activation or release of intracellular calcium by about 10%, about 20%, about 30%, about 40%, about 50%, about 60%, about 70%, about 80%, about 90%, or about 100%, compared to a neuron that does not express the excitatory DREADD. An excitatory DREADD can increase activation or release of intracellular calcium from about 10% to about 100%, about 20% to about 90%, about 30% to about 80%, about 40% to about 70%, or about 50% to about 60% compared to a neuron that does not express the excitatory DREADD.

In some embodiments, activation of a neuron comprises activation of a glutamatergic neuron, a GABAergic neuron, a dopaminergic neuron, or a serotonin neuron. In some embodiments, the neuron comprises a neuron of the inferior colliculus. In some embodiments, activation of the neuron comprises activation of a neuron of the caudate nucleus. In some embodiments, activation of the neuron comprises activation of a neuron of the left caudate nucleus. In some embodiments, activation of the neuron comprises activation of a neuron of the right caudate nucleus.

In some embodiments, a symptom of a neurological disease and/or condition in a subject is reduced when a neuron of a subject is activated. The symptom of the subject can be reduced by about 10%, about 20%, about 30%, about 40%, about 50%, about 60%, about 70%, about 80%, about 90%, or about 100% compared to a subject that does not comprise the activated neuron. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated neuron. In some embodiments, the dizziness is reduced by about 10%, about 20%, about 30%, about 40%, about 50%, about 60%, about 70%, about 80%, about 90%, or about 100% compared to a subject that does not comprise an activated neuron. In some embodiments, the hearing impairment is reduced by about 10%, about 20%, about 30%, about 40%, about 50%, about 60%, about 70%, about 80%, about 90%, or about 100% compared to a subject that does not comprise the activated neuron. In some embodiments, the buzzing is reduced by about 10%, about 20%, about 30%, about 40%, about 50%, about 60%, about 70%, about 80%, about 90%, or about 100% compared to a subject that does not comprise the activated neuron. In some embodiments, the clicking is reduced by about 10%, about 20%, about 30%, about 40%, about 50%, about 60%, about 70%, about 80%, about 90%, or about 100% compared to a subject that does not comprise the activated neuron. In some embodiments, the humming is reduced by about 10%, about 20%, about 30%, about 40%, about 50%, about 60%, about 70%, about 80%, about 90%, or about 100% compared to a subject that does not comprise the activated neuron. In some embodiments, the hissing is reduced by about 10%, about 20%, about 30%, about 40%, about 50%, about 60%, about 70%, about 80%, about 90%, or about 100% compared to a subject that does not comprise the activated neuron. In some embodiments, the roaring is reduced by about 10%, about 20%, about 30%, about 40%, about 50%, about 60%, about 70%, about 80%, about 90%, or about 100% compared to a subject that does not comprise the activated neuron. In some embodiments, the ringing is reduced by about 10%, about 20%, about 30%, about 40%, about 50%, about 60%, about 70%, about 80%, about 90%, or about 100% compared to a subject that does not comprise the activated neuron.

In some embodiments, a symptom of a neurological disease and/or condition in a subject is reduced when a small molecule actuator is introduced to a glutamatergic neuron of a subject, thereby comprising an activated glutamatergic neuron. The symptom of the subject can be reduced by about 10%, about 20%, about 30%, about 40%, about 50%, about 60%, about 70%, about 80%, about 90%, or about 100% compared to a subject that does not comprise the activated glutamatergic neuron. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated glutamatergic neuron. In some embodiments, the dizziness is reduced by about 10%, about 20%, about 30%, about 40%, about 50%, about 60%, about 70%, about 80%, about 90%, or about 100% compared to a subject that does not comprise the activated glutamatergic neuron. In some embodiments, the hearing impairment is reduced by about 10%, about 20%, about 30%, about 40%, about 50%, about 60%, about 70%, about 80%, about 90%, or about 100% compared to a subject that does not comprise the activated glutamatergic neuron. In some embodiments, the buzzing is reduced by about 10%, about 20%, about 30%, about 40%, about 50%, about 60%, about 70%, about 80%, about 90%, or about 100% compared to a subject that does not comprise the activated glutamatergic neuron. In some embodiments, the clicking is reduced by about 10%, about 20%, about 30%, about 40%, about 50%, about 60%, about 70%, about 80%, about 90%, or about 100% compared to a subject that does not comprise the activated glutamatergic neuron. In some embodiments, the humming is reduced by about 10%, about 20%, about 30%, about 40%, about 50%, about 60%, about 70%, about 80%, about 90%, or about 100% compared to a subject that does not comprise the activated glutamatergic neuron. In some embodiments, the hissing is reduced by about 10%, about 20%, about 30%, about 40%, about 50%, about 60%, about 70%, about 80%, about 90%, or about 100% compared to a subject that does not comprise the activated glutamatergic neuron. In some embodiments, the roaring is reduced by about 10%, about 20%, about 30%, about 40%, about 50%, about 60%, about 70%, about 80%, about 90%, or about 100% compared to a subject that does not comprise the activated glutamatergic neuron. In some embodiments, the ringing is reduced by about 10%, about 20%, about 30%, about 40%, about 50%, about 60%, about 70%, about 80%, about 90%, or about 100% compared to a subject that does not comprise the activated glutamatergic neuron.

In some embodiments, a symptom of a neurological disease and/or condition in a subject is reduced when a small molecule actuator is introduced to a GABAergic neuron of a subject, thereby activating the GABAergic neuron. The symptom of the subject can be reduced by about 10%, about 20%, about 30%, about 40%, about 50%, about 60%, about 70%, about 80%, about 90%, or about 100% compared to a subject that does not comprise the activated GABAergic neuron. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated GABAergic neuron. In some embodiments, the dizziness is reduced by about 10%, about 20%, about 30%, about 40%, about 50%, about 60%, about 70%, about 80%, about 90%, or about 100% compared to a subject that does not comprise the activated GABAergic neuron. In some embodiments, the hearing impairment is reduced by about 10%, about 20%, about 30%, about 40%, about 50%, about 60%, about 70%, about 80%, about 90%, or about 100% compared to a subject that does not comprise the activated GABAergic neuron. In some embodiments, the buzzing is reduced by about 10%, about 20%, about 30%, about 40%, about 50%, about 60%, about 70%, about 80%, about 90%, or about 100% compared to a subject that does not comprise the activated GABAergic neuron. In some embodiments, the clicking is reduced by about 10%, about 20%, about 30%, about 40%, about 50%, about 60%, about 70%, about 80%, about 90%, or about 100% compared to a subject that does not comprise the activated GABAergic neuron. In some embodiments, the humming is reduced by about 10%, about 20%, about 30%, about 40%, about 50%, about 60%, about 70%, about 80%, about 90%, or about 100% compared to a subject that does not comprise the activated GABAergic neuron. In some embodiments, the hissing is reduced by about 10%, about 20%, about 30%, about 40%, about 50%, about 60%, about 70%, about 80%, about 90%, or about 100% compared to a subject that does not comprise the activated GABAergic neuron. In some embodiments, the roaring is reduced by about 10%, about 20%, about 30%, about 40%, about 50%, about 60%, about 70%, about 80%, about 90%, or about 100% compared to a subject that does not comprise the activated GABAergic neuron. In some embodiments, the ringing is reduced by about 10%, about 20%, about 30%, about 40%, about 50%, about 60%, about 70%, about 80%, about 90%, or about 100% compared to a subject that does not comprise the activated GABAergic neuron.

In some embodiments, a symptom of a neurological disease and/or condition in a subject is reduced when a small molecule actuator is introduced to a dopaminergic neuron of a subject, thereby activating the dopaminergic neuron. The symptom of the subject can be reduced by about 10%, about 20%, about 30%, about 40%, about 50%, about 60%, about 70%, about 80%, about 90%, or about 100% compared to a subject that does not comprise the activated dopaminergic neuron. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated dopaminergic neuron. In some embodiments, the dizziness is reduced by about 10%, about 20%, about 30%, about 40%, about 50%, about 60%, about 70%, about 80%, about 90%, or about 100% compared to a subject that does not comprise the activated dopaminergic neuron. In some embodiments, the hearing impairment is reduced by about 10%, about 20%, about 30%, about 40%, about 50%, about 60%, about 70%, about 80%, about 90%, or about 100% compared to a subject that does not comprise the activated dopaminergic neuron. In some embodiments, the buzzing is reduced by about 10%, about 20%, about 30%, about 40%, about 50%, about 60%, about 70%, about 80%, about 90%, or about 100% compared to a subject that does not comprise the activated dopaminergic neuron. In some embodiments, the clicking is reduced by about 10%, about 20%, about 30%, about 40%, about 50%, about 60%, about 70%, about 80%, about 90%, or about 100% compared to a subject that does not comprise the activated dopaminergic neuron. In some embodiments, the humming is reduced by about 10%, about 20%, about 30%, about 40%, about 50%, about 60%, about 70%, about 80%, about 90%, or about 100% compared to a subject that does not comprise the activated dopaminergic neuron. In some embodiments, the hissing is reduced by about 10%, about 20%, about 30%, about 40%, about 50%, about 60%, about 70%, about 80%, about 90%, or about 100% compared to a subject that does not comprise the activated dopaminergic neuron. In some embodiments, the roaring is reduced by about 10%, about 20%, about 30%, about 40%, about 50%, about 60%, about 70%, about 80%, about 90%, or about 100% compared to a subject that does not comprise the activated dopaminergic neuron. In some embodiments, the ringing is reduced by about 10%, about 20%, about 30%, about 40%, about 50%, about 60%, about 70%, about 80%, about 90%, or about 100% compared to a subject that does not comprise the activated dopaminergic neuron.

In some embodiments, a symptom of a neurological disease and/or condition in a subject is reduced when a small molecule actuator is introduced to a serotonin neuron of a subject, thereby activating the serotonin neuron. The symptom of the subject can be reduced by about 10%, about 20%, about 30%, about 40%, about 50%, about 60%, about 70%, about 80%, about 90%, or about 100% compared to a subject that does not comprise the activated serotonin neuron. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated serotonin neuron. In some embodiments, the dizziness is reduced by about 10%, about 20%, about 30%, about 40%, about 50%, about 60%, about 70%, about 80%, about 90%, or about 100% compared to a subject that does not comprise the activated serotonin neuron. In some embodiments, the hearing impairment is reduced by about 10%, about 20%, about 30%, about 40%, about 50%, about 60%, about 70%, about 80%, about 90%, or about 100% compared to a subject that does not comprise the activated serotonin neuron. In some embodiments, the buzzing is reduced by about 10%, about 20%, about 30%, about 40%, about 50%, about 60%, about 70%, about 80%, about 90%, or about 100% compared to a subject that does not comprise the activated serotonin neuron. In some embodiments, the clicking is reduced by about 10%, about 20%, about 30%, about 40%, about 50%, about 60%, about 70%, about 80%, about 90%, or about 100% compared to a subject that does not comprise the activated serotonin neuron. In some embodiments, the humming is reduced by about 10%, about 20%, about 30%, about 40%, about 50%, about 60%, about 70%, about 80%, about 90%, or about 100% compared to a subject that does not comprise the activated serotonin neuron. In some embodiments, the hissing is reduced by about 10%, about 20%, about 30%, about 40%, about 50%, about 60%, about 70%, about 80%, about 90%, or about 100% compared to a subject that does not comprise the activated serotonin neuron. In some embodiments, the roaring is reduced by about 10%, about 20%, about 30%, about 40%, about 50%, about 60%, about 70%, about 80%, about 90%, or about 100% compared to a subject that does not comprise the activated serotonin neuron. In some embodiments, the ringing is reduced by about 10%, about 20%, about 30%, about 40%, about 50%, about 60%, about 70%, about 80%, about 90%, or about 100% compared to a subject that does not comprise the activated serotonin neuron.

In some embodiments, a symptom of a neurological disease and/or condition in a subject is reduced when a small molecule actuator is introduced to a neuron of the caudate nucleus of a subject, thereby activating the neuron of the caudate nucleus. The symptom of the subject can be reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the dizziness is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the hearing impairment is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the buzzing is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the clicking is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the humming is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the hissing is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the roaring is reduced by about 10%, about 20%, about 30%, about 40%, about 50%, about 60%, about 70%, about 80%, about 90%, or about 100% compared to a subject that does not comprise the activated neuron of the caudate nucleus. In some embodiments, the ringing is reduced by about 10%, about 20%, about 30%, about 40%, about 50%, about 60%, about 70%, about 80%, about 90%, or about 100% compared to a subject that does not comprise the activated neuron of the caudate nucleus.

In some embodiments, a symptom of a neurological disease and/or condition in a subject is reduced when a small molecule actuator is introduced to a neuron of the left caudate nucleus of a subject, thereby activating the neuron of the left caudate nucleus. The symptom of the subject can be reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the dizziness is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the hearing impairment is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the buzzing is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the clicking is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the humming is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the hissing is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the roaring is reduced by about 10%, about 20%, about 30%, about 40%, about 50%, about 60%, about 70%, about 80%, about 90%, or about 100% compared to a subject that does not comprise the activated neuron of the left caudate nucleus. In some embodiments, the ringing is reduced by about 10%, about 20%, about 30%, about 40%, about 50%, about 60%, about 70%, about 80%, about 90%, or about 100% compared to a subject that does not comprise the activated neuron of the left caudate nucleus.

In some embodiments, a symptom of a neurological disease and/or condition in a subject is reduced when a small molecule actuator is introduced to a neuron of the right caudate nucleus of a subject. The symptom of the subject can be reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the symptom comprises vertigo, dizziness, hearing impairment, buzzing, clicking, humming, hissing, roaring, or ringing. In some embodiments, the vertigo is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the dizziness is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the hearing impairment is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the buzzing is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the clicking is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the humming is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the hissing is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the roaring is reduced by about 10%, about 20%, about 30%, about 40%, about 50%, about 60%, about 70%, about 80%, about 90%, or about 100% compared to a subject that does not comprise the activated neuron of the right caudate nucleus. In some embodiments, the ringing is reduced by about 10%, about 20%, about 30%, about 40%, about 50%, about 60%, about 70%, about 80%, about 90%, or about 100% compared to a subject that does not comprise the activated neuron of the right caudate nucleus.

### Methods for Expressing Genes and Delivery to the Central Nervous System

In some embodiments, delivery of the gene therapy to a subject comprises intravenous (IV) injection, oral administration, intramuscular injection, subcutaneous injection, intrathecal therapy, rectal administration, vaginal administration, or inhalation.

The nervous system is organized into two main parts, the central nervous system (CNS) and the peripheral nervous system (PNS). The CNS is the processing center of the body and consists of the brain and the spinal cord. Accessing the CNS can require a therapy to bypass the blood-brain barrier. Strategies to deliver therapeutics to the CNS include, but are not limited to, intra-arterial chemotherapy, direct injection of therapeutic substances into intracranial lesions, and use of nanoparticles for drug delivery.

In certain embodiments, the compositions and/or pharmaceutical compositions of the present disclosure can be injected into a subject. In certain embodiments, injections can be given directly into the brain. In some embodiments, the pharmaceutical composition is directly injected into the striatum, which is a nucleus in the subcortical basal ganglia of the forebrain. The compositions and/or pharmaceutical compositions can be injected directly into the IC, the Caudate Nucleus, the right Caudate Nucleus, and/or the left Caudate Nucleus.

In some embodiments, the injected gene can target a specific type of neurons or set of neurons in the brain. In some embodiments, the neuron can be a neuron of the striatum, the IC, the Caudate Nucleus, the right Caudate Nucleus, and/or the left Caudate Nucleus. In some embodiments, the neuron can be a medium spiny neuron. In some embodiments, the neuron can be a dopaminergic medium spiny neuron. In some embodiments the dopaminergic medium spiny neuron comprises a D1 dopaminergic medium spiny neuron. In some embodiments, the neuron comprises a glutamatergic neuron, a GABAergic neuron, and/or a serotonin neuron. In some embodiments, the neuron can be a combination of neurons disclosed herein.

Injections can be performed using stereotactic surgery or intracerebral injections. Stereotactic surgery is a minimally invasive form of surgical intervention that makes use of a three-dimensional coordinate system to locate small targets inside the body and to perform on them some action such as ablation, biopsy, lesion, injection, stimulation, implantation, radiosurgery (SRS), etc. Intracerebral injection, such as intracerebroventricular injection, is an invasive injection technique of substances directly into the cerebrospinal fluid in cerebral ventricles in order to bypass the blood-brain barrier.

In some embodiments, the genetic therapy of the disclosure is used to genetically engineer a neuron. In some embodiments the genetic engineering method results in the expression of the polypeptide by the neuron. In some embodiments, the peptide expressed by the neuron is translated to form a therapeutic protein. In some embodiments, the therapeutic protein delivered by the methods and compositions of the present disclosure is useful for targeting and treating a neurodegenerative disease and/or condition. In certain embodiments, described herein is a method of preparing a disease treatment comprising administering one or more pharmaceutically acceptable excipients, carriers, or diluents and a composition and/or pharmaceutical composition of the present disclosure.

As described in WO2008096268, in gene therapy embodiments employing viral delivery of a composition disclosed herein, the unit dose can be calculated in terms of the dose of viral particles being administered. Viral doses include a particular number of virus particles or plaque forming units (pfu). For embodiments involving adenovirus, particular unit doses include 103, 104, 105, 106, 107, 108, 109, 1010, 1011, 1012, 1013 or 1014 pfu. Particle doses can be somewhat higher (10 to 100 fold) due to the presence of infection-defective particles.

In some embodiments, a composition of the present disclosure is delivered to the subject by a non-viral method. In some examples, the non-viral method comprises lipofection, nanoparticle delivery, particle bombardment, electroporation, sonication or microinjection.

In some embodiments, the gene therapy is administered to a subject with a volume of from about 1 nl to about 1000 nl. In some embodiments, the volume comprises from about 1 nl to about 100 nl, from about 1 nl to about 200 nl, from about 1 nl to about 300 nl, from about 1 nl to about 400 nl, from about 1 nl to about 500 nl, from about 1 nl to about 600 nl, from about 1 nl to about 700 nl, from about 1 nl to about 800 nl, from about 1 nl to about 900 nl, from about 1 nl to about 1000 nl.

In some embodiments, the AAV vector of the present disclosure is administered to a subject at a viral titer from about 1×10¹⁴ vg/kg to about 3×10¹⁴ vg/kg. In some embodiments, the viral titer comprises from about 1×10¹⁴ vg/kg to about 2×10¹⁴ vg/kg, about 1×10¹⁴ vg/kg to about 1.5×10¹⁴ vg/kg, about 1.5×10¹⁴ vg/kg to about 2×10¹⁴ vg/kg, about 2.5×10¹⁴ vg/kg to about 3×10¹⁴ vg/kg, or about 1.5×10¹⁴ vg/kg to about 2.5×10¹⁴ vg/kg.

### Compositions

In some embodiments, the compositions and methods of treating diseases and/or conditions disclosed herein comprise DREADDs and the administration of DREADDS and small molecule actuators to a subject. In some embodiments, the following compositions can be used in any combination. The compositions can comprise a vector, a promoter, a gene of interest, and an expression marker. In some embodiments, one having skill in the art can combine a cell type specific promoter disclosed below with a suitable vector, such as an AAV described below, a suitable gene of interest, such as a gene of interest described below, marker. In some embodiments, a composition can comprise a cell specific promoter for an inhibitory neuron, such as a Vgat promoter, operatively coupled to an inhibitory or excitatory DREADD. In some embodiments, a composition of the present disclosure can comprise a cell specific promoter for an excitatory neuron, such as a CamKII promoter, operatively coupled to an inhibitory or excitatory DREADD. In some embodiments, the compositions and methods described herein can comprise both an inhibitory and excitatory DREADD, with promoters targeting the same cell, or different cells.

In some embodiments, a vector of the composition can comprise AAV1, AAV1, AAV2, AAV3, AAV4, AAV5, AAV6, AAV7, AAV8, AAV9, AAV10, AAV11, AAV12, AAV13, Rh10, PHP.B, PHP.eB, or PHP.S.

In some embodiments, a promoter of the composition can comprise a synapsin I (SYN) promoter (e.g., hSYN1), a calcium/calmodulin-dependent protein kinase II (CamKII) promoter, a vesicular GABA transporter (Vgat) promoter, a tubulin alpha I, a neuron-specific enolase, a platelet-derived growth factor beta chain promoter, an astrocyte-specific glial fibrillary acidic protein (GFAP) promoter, a cerebellar Purkinje cell-specific L7-6 promoter, a dopamine receptor D1 (DRD1) promoter, a dopamine receptor D2 (DRD2) promoter, a parvalbumin (Pvalb) promoter, or a distal-less homeobox (Dlx) promoter.

In some embodiments, a gene of interest of the composition can comprise a Gi-DREADD, a Gq-DREADD, a Gs-DREADD, an Arrestin-DREADD, an Axonally-targeted silencing DREADD, or a KORD-DREADD.

In some embodiments, an expression marker of the composition can comprise a fluorescent protein. The fluorescent protein can comprise a red, blue, green, or yellow fluorescent protein, or any other suitable fluorescent protein.

In some embodiments, a small molecule actuator can be administered to a subject following the administration of a DREADD to a subject, as described herein.

### Combination Therapy

In some embodiments the methods, compositions and/or gene therapy constructs of the present disclosure can be comprise an additional therapeutic agent. The additional therapeutic agent can comprise one or more sodium channel blockers, antioxidants, spin-trapping agents, N-methyl-D-aspartate (NMDA) antagonists, selective serotonin reuptake inhibitor (SSRI) antagonists, dopamine releasing agents (DRA), acetylcholine release inducers, norepinephrine reuptake inhibitors (NERI), monoaminoxidase-A inhibitors (MAI), serotonin reuptake inhibitors (SRI), serotonin-norepinephrine reuptake inhibitors (SNRI), norepinephrine selective reuptake inhibitors (NSRI), serotonin reuptake inhibitors (5HT SRI), zonisamide, gabapentin, cannabinoid, a tetracycline promoter and tetracycline, or any combinations thereof.

### Pharmaceutical Compositions

In some embodiments the methods, compositions and/or gene therapy constructs of the present disclosure are included in a pharmaceutical composition comprising one or more pharmaceutically acceptable excipients, carriers, stabilizers, dispersing agents, suspending agents, thickening agents, and/or diluents. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, optic, subcutaneous, transdermal, nasal, intravitreal, intratracheal, intrapulmonary, transmucosal, vaginal, and topical administration.

Formulations can be modified depending upon the route of administration chosen. Pharmaceutical compositions comprising a compound described herein can be manufactured, for example, by mixing, dissolving, emulsifying, encapsulating, entrapping, or compression processes.

Pharmaceutical compositions can be formulated by combining the active compounds with pharmaceutically-acceptable carriers or excipients. Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof. Pharmaceutically acceptable excipients include, but are not limited to, liquids such as water, saline, glycerol and ethanol. Pharmaceutically acceptable salts such as inorganic acid salts such as hydrochloride, bromate, phosphate, sulfate, etc.; And salts of organic acids such as acetates, propionates, malonates, benzoates, etc. can be included in pharmaceutical compositions. Additionally, reinforcing materials such as wetting or emulsifying agents, pH buffering materials, and the like can be present in such vehicles. A wide variety of pharmaceutically acceptable excipients are known in the art and need not be discussed in detail herein. Pharmaceutically acceptable excipients are, for example, A. Gennaro (2000) “Remington: The Science and Practice of Pharmacy,” 20th edition, Lippincott, Williams, &Wilkins; Pharmaceutical Dosage Forms and Drug Delivery Systems (1999) H. C. Ansel et al., Eds., 7th ed., Lippincott, Williams, &Wilkins; and Handbook of Pharmaceutical Excipients (2000) A. H. Kibbe et al., Eds., 3rd ed. Amer.

Non-limiting examples of pharmaceutically-acceptable carriers include saline solution, Ringer's solution and dextrose solution. In some embodiments, the carrier includes sustained release preparations such as semipermeable matrices of solid hydrophobic polymers containing the compound disclosed herein, where the matrices are in the form of shaped articles, such as films, liposomes, microparticles, and microcapsules.

Carbomers in an aqueous pharmaceutical composition serve as emulsifying agents and viscosity modifying agents. In certain embodiments, the pharmaceutically acceptable excipient comprises or consists of a carbomer. In certain embodiments, the carbomer comprises or consists of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents. In certain embodiments, the pharmaceutically acceptable excipient comprises or consists of a cyclodextrin. In certain embodiments, the cyclodextrin comprises or consists of alpha cyclodextrin, beta cyclodextrin, gamma cyclodextrin, or combinations thereof. Lecithin in a pharmaceutical composition serve as a solubilizing agent. In certain embodiments, the solubilizing agent comprises or consists of lecithin. Poloxamers in a pharmaceutical composition serve as emulsifying agents, solubilizing agents, and dispersing agents. In certain embodiments, the pharmaceutically acceptable excipient comprises or consists of a poloxamer. In certain embodiments, the poloxamer comprises or consists of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. In certain embodiments, the pharmaceutically acceptable excipient comprises or consists of a polyoxyethylene sorbitan fatty acid ester. In certain embodiments, the polyoxyethylene sorbitan fatty acid ester comprises or consists of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. In certain embodiments, the pharmaceutically acceptable excipient comprises or consists of a polyoxyethylene stearate. In certain embodiments, the polyoxyethylene stearate comprises or consists of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. In certain embodiments, the pharmaceutically acceptable excipient comprises or consists of a sorbitan ester. In certain embodiments, the sorbitan ester comprises or consists of sorbitan laurate, sorbitan oleate, sorbitan palmitate, sorbitan stearate, sorbitan trioleate, sorbitan sesquioleate, or combinations thereof. In certain embodiments, solubility can be achieved with a protein carrier. In certain embodiments the protein carrier comprises albumin, human albumin.

In certain embodiments a polypeptide can be stabilized by polyuronides. In certain embodiments, the stabilizer comprises or consists of a polyuronide. In certain embodiments, the polyuronide comprises or consists of calcium alginate.

In certain embodiments, the compositions and/or gene therapy constructs of the present disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the compositions and/or gene therapy constructs of the present disclosure are shipped/stored lyophilized and reconstituted before administration. In certain embodiments, lyophilized compositions and/or gene therapy constructs formulations comprise a bulking agent such as, mannitol, sorbitol, sucrose, trehalose, dextran 40, or combinations thereof. The lyophilized formulation can be contained in a vial comprised of glass or other suitable non-reactive material. The compositions and/or gene therapy constructs when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

A pharmaceutical composition can be administered in a local or systemic manner, for example, via injection of the compound directly into an organ, optionally in a depot or sustained release formulation or implant. Pharmaceutical compositions can be provided in the form of a rapid release formulation, in the form of an extended-release formulation, or in the form of an intermediate release formulation. A rapid release form can provide an immediate release. An extended release formulation can provide a controlled release or a sustained delayed release.

In practicing the methods of treatment or use provided herein, therapeutically-effective amounts of the compounds described herein are administered in pharmaceutical compositions to a subject having a disease or condition to be treated. A therapeutically-effective amount can vary widely depending on the severity of the disease, the age and relative health of the subject, the potency of the compounds used, and other factors. The compounds can be used singly or in combination with one or more therapeutic agents as components of mixtures.

In some embodiments, the pharmaceutical administration is given to an animal including but not limited to a vertebrate, such as a mammal, avian or fish. An animal can be a human or a bovine, canine, caprine, cervine, cricetine, feline, galline, equine, lapine, murine, musteline and ovine. An animal can be a human or other mammalian animals including primates (e.g., monkeys), bovine (e.g., cattle or dairy cows), porcine (e.g., hogs or pigs), ovine (e.g., goats or sheep), equine (e.g., horses), canine (e.g., dogs), feline (e.g., house cats), antelopes, buffalos, camels, cervine (e.g., deer), donkeys, rabbits, and rodents (e.g., guinea pigs, squirrels, rats, mice, gerbils, and hamsters). In some embodiments, the pharmaceutical administration is administered to a human.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, or between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

### Methods of Treatment

In some embodiments, a method of treatment comprises genetically engineering or modifying a neuron of a subject comprising administering a composition disclosed herein, wherein the composition comprises a DREADD configured for expression in a particular population of cells. In some embodiments, the method comprises administering the composition to a cell comprising an inhibitory or excitatory neuron and activating the inhibitory or excitatory neuron by administering a small molecule actuator disclosed herein. In some embodiments, the method comprises activating a inhibitory neuron comprising an excitatory or inhibitory DREADD in one area of the brain, for example the auditory cortex, inferior colliculus, hippocampus, caudate nucleus, left caudate nucleus, or right caudate nucleus, and activating an excitatory neuron comprising an excitatory or inhibitory DREADD in the same or a different area of the brain, for example the auditory cortex, inferior colliculus, hippocampus, caudate nucleus, left caudate nucleus, or right caudate nucleus.

Diseases and/or Conditions

In some embodiments, the compositions and methods disclosed herein can be used to treat a disease/disorder or condition. In some embodiments, the disease/disorder or condition comprises a neurological disease/disorder or condition. In some embodiments, the neurological disease/disorder or condition comprises an auditory neurological disease/disorder or condition. In some embodiments, the auditory neurological disease/disorder or condition comprises tinnitus or Meniere's disease, noise induced hearing loss (NIHL), sensorineural hearing loss, auditory neuropathy spectrum disorder, and/or central auditory processing disorder.

Other non-limiting examples of neurological diseases or disorders that can be treated by the compositions and methods herein include: Abulia, Agraphia, Alcoholism, Alexia, Alzheimer's disease (AD) Aneurysm, Amaurosis fugax, Amnesia, Amyotrophic lateral sclerosis (ALS), Angelman syndrome, Aphasia, Apraxia, Arachnoiditis, Arnold-Chiari malformation, Asperger syndrome, Ataxia, Ataxia-telangiectasia, Attention deficit hyperactivity disorder, Auditory processing disorder, Autism spectrum, Bipolar disorder, Bell's palsy, Brachial plexus injury, Brain damage, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy (CADASIL), Cerebral gigantism, Cerebral palsy, Cerebral vasculitis, Cervical spinal stenosis, Charcot-Marie-Tooth disease, Chiari malformation, Chorea, Chronic fatigue syndrome, Chronic inflammatory demyelinating polyneuropathy (CIDP), Chronic pain, Coffin “Lowry syndrome, Coma, Complex regional pain syndrome, Compression neuropathy, Congenital facial diplegia, Corticobasal degeneration, Cranial arteritis, Craniosynostosis, Creutzfeldt-Jakob disease, Cumulative trauma disorders, Cushing's syndrome, Cyclothymic disorder, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Developmental coordination disorder, Diabetic neuropathy, Diffuse sclerosis, Diplopia, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalotrigeminal angiomatosis, Encopresis, Enuresis, Epilepsy, Epilepsy-intellectual disability in females, Erb's palsy, Erythromelalgia, Exploding head syndrome, Fabry's disease, Fahr's syndrome, Fainting, Familial spastic paralysis, Febrile seizures, Fisher syndrome, Friedreich's ataxia, Fibromyalgia, Foville's syndrome, Fetal alcohol syndrome, Fragile X syndrome, Fragile X-associated tremor/ataxia syndrome (FXTAS), Gaucher's disease, Generalized epilepsy with febrile seizures plus, Gerstmann's syndrome, Giant cell arteritis, Giant cell inclusion disease, Globoid Cell Leukodystrophy, Gray matter heterotopia, Guillain-Barre syndrome, Generalized anxiety disorder, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Hirschsprung's disease, Holmes-Adie syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile Refsum disease, Infantile spasms, Inflammatory myopathy, Intracranial cyst, Intracranial hypertension, Isodicentric 15, Joubert syndrome, Karak syndrome, Kearns-Sayre syndrome, Kinsbourne syndrome, Kleine-Levin Syndrome, Klippel Feil syndrome, Krabbe disease, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-In syndrome, Lumbar disc disease, Lumbar spinal stenosis, Lyme disease-Neurological Sequelae, Machado-Joseph disease (Spinocerebellar ataxia type 3), Macrencephaly, Macropsia, Mal de debarquement, Megalencephalicleukoencephalopathy with subcortical cysts, Megalencephaly, Melkersson-Rosenthal syndrome, Meniere's disease, Meningitis, Menkes disease, Metachromaticleukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Motor skills disorder, Moyamoya disease, Mucopolysaccharidoses, Multi-infarct dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-Behcet's disease, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Neuropathy, Neurosis, Niemann-Pick disease, Non-hour sleep” wake disorder, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Otosclerosis, Overuse syndrome, Palinopsia, Paresthesia, Parkinson's disease, Paramyotonia Congenita, Paraneoplastic diseases, Paroxysmal attacks, Parry-Romberg syndrome, PANDAS, Pelizaeus-Merzbacher disease, Periodic Paralyses, Peripheral neuropathy, Pervasive developmental disorders, Photic sneeze reflex, Phytanic acid storage disease, Pick's disease, Pinched nerve, Pituitary tumors, PMG, Polyneuropathy, Polio, Polymicrogyria, Polymyositis, Porencephaly, Post-Polio syndrome, Postherpetic Neuralgia (PHN), Postural Hypotension, Prader-Willi syndrome, Primary Lateral Sclerosis, Prion diseases, Progressive hemifacial atrophy, Progressive multifocal leukoencephalopathy, Progressive Supranuclear Palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic Movement Disorder, Romberg syndrome, Saint Vitus dance, Sandhoff disease, Schilder's disease, Schizencephaly, Sensory processing disorder, Septo-optic dysplasia, Shaken baby syndrome, Shingles, Shy-Drager syndrome, Sjogren's syndrome, Sleep apnea, Sleeping sickness, Snatiation, Sotos syndrome, Spasticity, Spina bifida, Spinal cord injury, Spinal cord tumors, Spinal muscular atrophy, Spinal and bulbar muscular atrophy, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tarsal tunnel syndrome, Tardive dyskinesia, Tardive dysphrenia, Tarlov cyst, Tay-Sachs disease, Temporal arteritis, Temporal lobe epilepsy, Tetanus, Tethered spinal cord syndrome, Thomsen disease, Thoracic outlet syndrome, Tic Douloureux, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Von Hippel-Lindau disease (VHL), Viliuisk Encephalomyelitis (VE), Wallenberg's syndrome, West syndrome, Whiplash, Williams syndrome, Wilson's disease, or Zellweger syndrome.

### LIST OF ENUMERATED EMBODIMENTS

A composition for treating a neurological disease or condition, the composition comprising: a vector, the vector comprising an adeno-associated virus (AAV) and a neuron specific promoter operatively coupled to a gene of interest.

A composition for treating a neurological disease or condition, the composition comprising: a first vector comprising a first AAV, and a first neuron specific promoter operatively coupled to a first gene of interest, and a second vector comprising a second AAV, and a second neuron specific promoter operatively coupled to a second gene of interest.

A composition comprising: a vector comprising a gene therapy construct, wherein the gene therapy construct comprises a heterologous nucleic acid encoding a designer receptor, wherein the designer receptor is configured to interact with a small molecule actuator, wherein the vector comprising the heterologous nucleic acid is configured to be expressed in a neuron, wherein expression of the heterologous nucleic acid modulates an inhibitory or an excitatory activity of the neuron, wherein the modulation results in a reduction of a symptom or presence of a disease or condition associated with the neuron.

A composition comprising a neuron, wherein the neuron comprises a heterologous nucleic acid encoding a protein configured to interact with a small molecule actuator, wherein the heterologous nucleic acid is configured to be expressed in a subject, wherein expression of the heterologous nucleic acid results in a reduction of a symptom of a neurological disease or condition when a small molecule actuator is administered to the subject.

The composition of any one of the preceding claims wherein the first or second vector comprises a nanoparticle.

The composition of any one of the preceding claims, wherein the neuron comprises a neuron of the auditory cortex, inferior colliculus, hippocampus, medial geniculate body, amygdala, and/or caudate nucleus.

The composition of any one of the preceding claims, wherein the neuron comprises a striatal neuron.

The composition of any one of the preceding claims, wherein the striatal neuron comprises a neuron of the caudate nucleus, the putamen, the nucleus accumbent, or a combination thereof.

The composition of any one of the preceding claims, wherein the striatal neuron comprises a neuron of the left caudate nucleus, the right caudate nucleus, or the left caudate nucleus and the right caudate nucleus.

The composition of any one of the preceding claims, wherein the neuron comprises an excitatory and/or inhibitory neuron.

The composition of any one of the preceding claims, wherein the neuron comprises a GABA ergic neuron or a glutamatergic neuron.

The composition of any one of the preceding claims, wherein the heterologous nucleic acid comprises one or more sequences configured to direct integration of the heterologous nucleic acid into a genomic location of a mammalian cell.

The composition of any one of the preceding claims, wherein the heterologous nucleic acid comprises a deoxyribonucleic acid (DNA).

The composition of any one of the preceding claims, wherein the heterologous nucleic acid comprises a ribonucleic acid (RNA).

The composition of any one of the preceding claims, wherein the heterologous nucleic acid comprises a nucleotide sequence comprising a promoter operatively coupled to an open reading frame of a gene of interest.

The composition of any one of the preceding claims, wherein the open reading frame of the gene of interest encodes a polypeptide.

The composition of any one of the preceding claims, wherein the promoter comprises a neuron specific promoter.

The composition of any one of the preceding claims, wherein the neuron specific promoter comprises a CamKII promoter, an hSYN promoter, or a Vgat promoter.

The composition of any one of the preceding claims, wherein the promoter comprises a bacterial PA, PR, and/or PR1/PR2 promoter.

The composition of any one of the preceding claims, wherein the gene of interest encodes a modified neuronal receptor.

The composition of any one of the preceding claims, wherein the gene of interest encodes a modified muscarinic receptor

The composition of any one of the preceding claims, wherein the gene of interest encodes a modified human muscarinic receptor.

The composition of any one of the preceding claims, wherein the modified muscarinic receptor comprises a designer receptor exclusively activated by designer drugs (DREADD).

The composition of claim 13, wherein the DREADD comprises one or more of hM3Ds, hM1Dq, hMD5q, hM4Di, or hM2Di, orhM3Dq.

The composition of claim 13, wherein the DREADD comprises an amino acid sequence exhibiting at least about 90%, 95%, 97%, 98%, 99% identity to or is identical to SEQ ID NO: 1.

The composition of any one of the preceding claims wherein the heterologous gene of interest is delivered to the cell using an adeno-associated virus (AAV).

The composition of any one of the preceding claims, wherein the AAV comprises AAV1, AAV2, AAV5, AAV8, AAV9 or AAV-DJ.

The composition of any one of the preceding claims, wherein the neurological disease or condition comprises an auditory disease or condition, such as tinnitus.

The composition of any one of the preceding claims, wherein the small molecule actuator comprises an atypical antipsychotic.

The composition of any one of the preceding claims, wherein the atypical antipsychotic comprises Clozapine-N-oxide.

The composition of any one of the preceding claims wherein the symptom of the auditory disease or condition comprises vertigo, dizziness, hearing impairment, buzzing, clicking, humming, hissing, roaring, or ringing.

The composition of any one of the preceding claims wherein the auditory disease or condition comprises tinnitus, Meniere's disease, noise induced hearing loss (NIHL), sensorineural hearing loss, auditory neuropathy spectrum disorder, and/or central auditory processing disorder.

A pharmaceutical composition comprising the heterologous nucleic acid of any one of the preceding claims and a pharmaceutically acceptable, carrier, excipient, and/or diluent.

The pharmaceutical composition of claim 33 wherein the pharmaceutical composition is formulated for delivery by direct injection to a brain of the subject.

The pharmaceutical composition of claim 34 wherein the pharmaceutical composition is formulated for delivery by nasal or cochlear injection to the subject.

The pharmaceutical composition of any one of the preceding claims, wherein the pharmaceutical formulation is formulated for delivery by lipid nanoparticles, liposomes, electroporation, plasmids, or non-viral delivery methods.

The pharmaceutical composition of any one of the preceding claims, wherein the non-viral delivery methods comprise, lipofection, nucleofection, microinjection, biolistics, virosomes, liposomes, immunoliposomes, polycation or lipid: nucleic acid conjugates, naked DNA, artificial virions, or agent-enhanced uptake of DNA.

A method of genetically engineering or modulating a neuron of a subject comprising administering the heterologous nucleic acid of any one of the preceding claims to the subject thereby genetically engineering the neuron.

The method of claim 37, wherein the neuron comprises a neuron of the auditory cortex, inferior colliculus, hippocampus, or caudate nucleus.

The method of claim 38 wherein the neuron comprises a striatal neuron.

The method of claim 37 or 38, wherein the striatal neuron comprises a neuron of the caudate nucleus, the putamen, the nucleus accumbent, or a combination thereof.

The method of any claims 37-40, wherein the striatal neuron comprises a neuron of the left caudate nucleus, the right caudate nucleus, or the left caudate nucleus and the right caudate nucleus.

The method of any one of the preceding claims, wherein the neuron comprises a medium-sized GABAergic spiny projection neuron or a glutamatergic neuron.

The method of any one of the preceding claims, wherein administering the heterologous nucleic acid comprises direct injection of the heterologous nucleic acid to the brain of a subject.

The method of any one of the preceding claims, wherein administering the heterologous nucleic acid comprises nasal and/or cochlear injection.

The method of any one of the preceding claims, wherein administering the heterologous nucleic acid comprises administering the heterologous nucleic acid using lipid nanoparticles, liposomes, electroporation, plasmids, or non-viral delivery methods to a subject.

The method of any one of the preceding claims, wherein the non-viral delivery methods comprise lipofection, nucleofection, microinjection, biolistics, virosomes, liposomes, immunoliposomes, polycation or lipid: nucleic acid conjugates, naked DNA, artificial virions, or agent-enhanced uptake of DNA.

The method of any one of the preceding claims, wherein the heterologous nucleic acid is administered at a dose of from about 1 nl to about 1000 nl.

The method of any one of the preceding claims, wherein the heterologous nucleic acid is administered at a viral titer of from about 1×10¹⁴ vg/kg to about 3×10¹⁴ vg/kg.

The method of any one of the preceding claims, wherein genetically engineering the neuron comprises expressing a heterologous G-protein coupled receptor (GPCR) in the neuron.

The method of any one of the preceding claims, wherein the heterologous GPCR comprises hM3Dq.

The method of any one of the preceding claims, wherein the heterologous GPCR comprises one or more of hM3Ds, hM1Dq, hMD5q, hM4Di, or hM2Di.

A method of treating a subject with a disease or condition comprising administering the heterologous nucleic acid of any one of the preceding claims or the pharmaceutical composition of any one of the preceding claims to a neuron of the subject with the disease or condition, expressing the heterologous nucleic acid as a protein configured to interact with a small molecule actuator, and activating the protein with the small molecule actuator, thereby treating the disease and/or condition.

The method of any one of the preceding claims, wherein the small molecule actuator comprises an atypical antipsychotic.

The method of any one of the preceding claims, wherein the atypical antipsychotic comprises Clozapine-N-oxide.

The method of any one of the preceding claims, wherein the disease or condition comprises an auditory disease or condition.

The method of any one of the preceding claims wherein the auditory disease or condition comprises tinnitus, Meniere's disease, noise induced hearing loss (NIHL), sensorineural hearing loss, auditory neuropathy spectrum disorder, and/or central auditory processing disorder.

The method of any one of the preceding claims, wherein the subject comprises a mammal.

The method of any one of the preceding claims, wherein the subject comprises a human.

The method of any one of the preceding claims, wherein the heterologous nucleic acid is administered to a neuron of the auditory cortex, inferior colliculus, hippocampus, medial geniculate body, amygdala, and/or caudate nucleus.

The method of any one of the preceding claims, wherein the heterologous nucleic acid is administered to the neurons of the left caudate nucleus.

The method of any one of the preceding claims, wherein the heterologous nucleic acid is administered to the neurons of the right caudate nucleus.

The method of any one of the preceding claims, wherein the heterologous nucleic acid is administered to the neurons of the right and left caudate nucleus.

The method of any one of the preceding claims, wherein the small molecule actuator is administered to the subject by injection.

The method of any one of the preceding claims, wherein the injection comprises intraperitoneal injection.

The method of any one of the preceding claims, wherein the small molecule actuator is administered to the subject at a dose of from about 0.5 mg/kg to about 20 mg/kg.

The method of any one of the preceding claims, wherein the small molecule actuator is administered to the subject once per day, twice per day, three times per day, four times per day, or more than four times per day.

The method of any one of the preceding claims, wherein the small molecule actuator is administered to the subject every 2 hours, every 3 hours, or every four hours.

The method of any one of the preceding claims, wherein the small molecule actuator is administered to the subject once per day, twice per day, or more than twice per day for at least one day, at least two days, at least 3 days, at least one week, at least two weeks, at least one month, or at least more than one month.

A method of treating a subject with a disease or condition comprising activating inhibitory or excitatory neurons in a brain region wherein the activating comprises administering the heterologous nucleic acid of any one of the preceding claims to an inhibitory or excitatory neuron within the brain region, expressing the protein encoded by the heterologous nucleic acid and activating the protein, thereby treating the disease or condition.

The method of any one of the preceding claims, wherein the brain region comprises the auditory cortex, inferior colliculus, hippocampus, medial geniculate body, amygdala, and/or caudate nucleus.

The method of any one of the preceding claims, wherein the inhibitory or excitatory neuron comprises a neuron of the inferior colliculus or the striatum.

The method of any one of the preceding claims, wherein the inhibitory or excitatory striatal neuron comprises a neuron of the caudate nucleus.

The method of any one of the preceding claims, wherein the inhibitory or excitatory striatal neuron comprises an inhibitory neuron of the left caudate nucleus, the right caudate nucleus, or the left caudate nucleus and the right caudate nucleus.

The method of any one of the preceding claims, wherein the inhibitory or excitatory neuron comprises a GABAergic neuron or a glutamatergic neuron.

The method of any one of the preceding claims, wherein the inhibitory neuron comprises a medium-sized GABAergic spiny projection neuron.

The method of any one of the preceding claims, wherein the disease or condition comprises an auditory disease or condition.

The method of any one of the preceding claims wherein the auditory disease or condition comprises tinnitus, Meniere's disease, noise induced hearing loss (NIHL), sensorineural hearing loss, auditory neuropathy spectrum disorder, and/or central auditory processing disorder.

The method of any one of the preceding claims, wherein the subject comprises a mammal.

The method of any one of the preceding claims, wherein the subject comprises a human.

The method of any one of the preceding claims, wherein the heterologous nucleic acid comprises an inhibitory or excitatory DREADD.

The method of any one of the preceding claims wherein the DREADD comprises the heterologous nucleic acid of any one of the preceding claims wherein the neuron specific promoter comprises a CamKII promoter, an hSYN promoter, or a Vgat promoter.

A method of activating the protein of any one of the preceding claims wherein the activating comprises administering an atypical antipsychotic to a subject.

The method of any one of the preceding claims, wherein the atypical antipsychotic comprises Clozapine-N-oxide.

The method of any one of the preceding claims comprising administering at least one additional composition or pharmaceutical composition to a subject.

The method of any one of the preceding claims, wherein the at least one additional composition or pharmaceutical composition comprises a therapeutic agent.

The method of any one of the preceding claims, wherein the therapeutic agent comprises a tetracycline promoter and tetracycline.

### EXAMPLES

These prophetic examples are provided for illustrative purposes only and not to limit the scope of the claims provided herein. It will be appreciated that variations in proportions and alternatives in elements of the components shown will be apparent to those skilled in the art and are within the scope of the embodiments presented herein.

### Example 1: Sound-Based Avoidance Model of Tinnitus

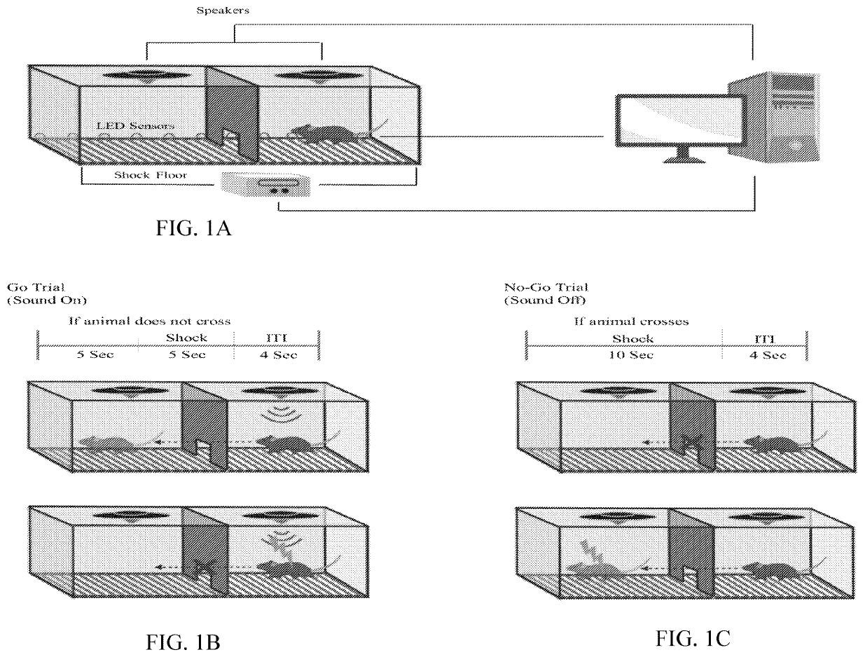

A sound-based avoidance detection (SBAD) method, as illustrated in FIG. 1, was used for detecting tinnitus and testing the animals' response to different pharmacological doses of NMDP. Using a shuttle box divided into two compartments, mice were trained to cross from side to side with sound cues in the “Go” trials and remain still in the absence of sound in the “No-Go” trials for 15 days. The animals had a 5-minute acclimation period in the shuttle box before testing began with 100 randomly assigned trials per day, lasting approximately 30-40 minutes. The sound cues were randomly played as white noise or narrow-band noise at 5, 10, 16, 20, and 32 kHz with sound intensities randomly at 75, 80, or 85 dB. To reinforce the training sessions, the mice were shocked if they did not cross from one compartment to the other during the Go trials, and as well in the No-Go trials if they moved compartments in the absence of sound. Additionally, a hurdle was added on training 11 to reinforce the training. After the 15th training day, mice were tested for 3 days to obtain a baseline score to be compared after noise exposure, as well as retraining for 3 days.

Following the successful completion of training so that mice reached a high percentage of success in the trials, the mice were exposed to a noise-induced trauma for tinnitus. To ensure that the mice still have their hearing, one ear was protected with an ear plug, while the other ear was exposed to the noise. The mice were placed in a sound booth where they were exposed to 120 dB broadband noise at 4-25 kHz for 2-4 hours. Following the noise exposure, the mice were housed for one month, and then tested to see if they have developed tinnitus. In these testing periods, the mice were tested for three days in the shuttle boxes, where they underwent the Go and No-Go trials, however in the No-Go trials the mice were not shocked, as it is expected that mice with tinnitus would cross compartments in the absence of a sound cue. The mice were continued to be shocked in the Go trials in order to ensure that they have retained their hearing and can hear the sound cues. In examining the results of the tests following noise exposure, tinnitus positive mice had a higher average of errors in the No-Go trials in comparison to their training. To insure there is a significant difference in the No-Go trial scores, chi-square tests were completed comparing the post-noise exposure tests to the baseline tests. However, if tinnitus is not present in the mice, tests were done after waiting one more month, and another if needed.

### Example 2: Chemogenetic Model of Tinnitus

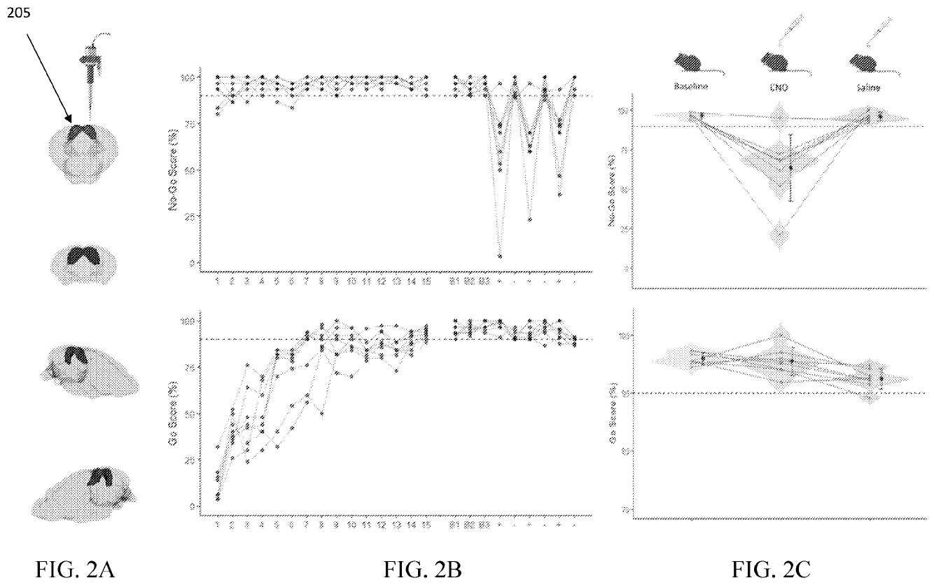

A chemogenetic-based model was established by using DREADDs to target excitatory neurons of the IC to establish a more reliable and efficient tinnitus model in mice. Stereotaxic microinjections of excitatory DREADDs [AAV8-CaMKIIa-HM3D-mCherry] were performed in these C57BL/6J mice, similar to previous studies (Saloman et al., 2016; Smith et al. 2016; Horii-Hayashi & Nishi 2021). Coordinates for microinjections were based on the atlas of Paxinos and Watson (2006) (AP: −5.00 mm, ML: +/−1.00 mm, DV: −1.50 mm). Mice were anesthetized with 5% isoflurane and placed in a stereotaxic head frame. During surgery mice were maintained at a 1.5-2% isoflurane level. Hair was removed and a vertical incision was made to expose the skull. The skull was leveled between bregma and lambda. Bilateral burr holes were drilled at the proper coordinates, and a glass pipette (50-85 μm) was lowered into the IC where 50 (n=4 mice) or 150 nL (n=4) of AAV was delivered at a rate of 2 nL/s. Gel cover was placed in the burr hole and the incision was closed using tissue adhesive. Ketoprofen (10 mg/kg) was administered to relieve pain and antibiotic ointment was applied to the incision site to prevent infection. Mice were given 1-month for postoperative recovery and completion of viral transduction. Following recovery, animals underwent SBAD training and baseline tests, followed by three SBAD tests post CNO IP injections and three SBAD tests post saline injections performed on alternate days. Of eight animals, seven (87%) tested positive for tinnitus one hour after receiving IP injections of CNO (chi-squared, p<0.03; FIG. 2). To rule out potential confounding stress factors related to IP injections, all animals received saline IP injections followed by SBAD testing one hour later. No-Go and Go scores remained stable compared to baseline scores. To rule out potential off-target effects of CNO that could affect SBAD testing, a control group of three SBAD trained animals received CNO IP injections (5 mg/kg) one hour before SBAD testing. Go and No-Go scores remained stable post-CNO injections, ruling out behavioral side effects of CNO. A 3×2 mixed analysis of variance (ANOVA) was computed using AAV dose (50 nL vs 150 nL) as the between-subjects factor and injectant type (CNO vs saline vs baseline) as the within-subjects factor. There was a significant main effect of injectant type [F (1, 6)=21.46, p<0.001, η2=0.66], but no significant main effect of AAV dose and no significant interaction between AAV dose and injectant type. Post-hoc t-tests (Bonferoni adjusted) indicated significantly reduced SBAD No-Go scores post-CNO injections compared to baseline and saline injections (p<. 001) and no significant difference between baseline and saline No-Go scores. Findings indicate successful induction of tinnitus using excitatory DREADDS with no difference in outcomes between 50 and 150 nL of AAV dose.

### Example 3: Chemogenetic Modulation in a Chemogenetic Tinnitus Model

Our proposed methodology will adopt the same surgical procedures discussed example 2, this time using different DREADD types, dosages and micro-injection targets. 50 C57BL6/J mice (4-6 weeks of age; 25 males) will first undergo stereotaxic surgery for bilateral 50 nL injections of AAV8-CaMKIIa-HM3D-mCherry into the central nucleus of the IC (AP: −5.00 mm, ML: +/−1.00 mm, DV: −1.50 mm) to induce tinnitus. Animals will be monitored daily for one month to ensure full recovery and to allow expression of excitatory DREADDs at targeted IC neurons. After surgical recovery, animals will undergo SBAD training and baseline testing over an estimated 18 days. Upon completion, animals will then receive IP injections of CNO (5 mg/kg) and saline (5 mg/kg) at alternate days for 6 consecutive days. SBAD testing will be completed one hour after each injection. Based on our preliminary work, we anticipate that approximately 45 of the animals will test positive for tinnitus post noise exposure. Animals that are considered tinnitus positive via CNO injections will undergo a second stereotaxic surgery for bilateral injections of either excitatory DREADDs (AAV2-Vgat-hM4D-mCherry; n=18)?, inhibitory DREADDs (AAV8-CaMKIIa-HM3D-mCherry; n=18), or control virus (AAV2-Vgat-mCherry; n=9) into the CN body (AP: +0.5 mm, ML: +/−1.75 mm, DV: −3 mm). Here, we again use the CaMKIIa promoter to target excitatory CN neurons and use vesicular GABA transporter (Vgat) to target CN GABAergic neurons, as Vgat are highly concentrated on the nerve endings of GABA neurons, as well as synaptic vesicles of glycinergic neurons (Chaudhry et al., 1998). Due to differences of neuronal population and distribution between our preliminary IC target and CN, we will deliver the excitatory and inhibitory DREADDs at three different doses bilaterally: 50 nL, 100 nL, and 150 nL (n=6 per dose for each DREADD type). Animals will then undergo a recovery period of 3-4 weeks. A SBAD retraining period of three to five days will be completed after recovery to ensure retention of task. Once SBAD retraining is completed with >90% Go and No-Go scores, postoperative SBAD testing procedures with CNO and saline IP injections will be repeated. Analyses will compare the efficacy of the DREADD type at the three administered doses to determine the appropriate neuronal target and AAV dose for the chemogenetic gene therapy. After the surgery, we will visualize co-localization of DREADDs in excitatory and inhibitory neurons by using immunofluorescence.

### Example 4: Chemogenetic Modulation in a Sound-Based Tinnitus Model

80 C57BL6/J mice (4 weeks of age) will complete SBAD training followed by baseline testing. Animals will then undergo noise-exposure to a broadband noise (4-25 kHz) at 120 dB SPL for two hours in a sound attenuating chamber. The right ear will be occluded during exposure to avoid bilateral noise induced hearing loss that would affect performance during Go trials. Hearing functions will be assessed pre- and post-noise exposure using tone burst evoked auditory brainstem response (ABR) thresholds (e.g., Akil et al., 2016) and distortion product otoacoustic emissions (DPOAE) (e.g., Martin et al., 2006) at frequencies 5-40 kHz at octave intervals. SBAD retesting will be completed two weeks, one month, and two months post noise exposure. Based on our previous work, we anticipate that approximately 40 of the animals will test positive for tinnitus post noise exposure. Animals that test positive for tinnitus will undergo stereotaxic surgery. A predetermined DREADD (AAV2-Vgat-hM3D-mCherry or AAV8-CaMKIIa-HM3D-mCherry) will be injected in the CN at either 50, 100 or 150 nL. DREADD type and dose selection will be based on findings from Aim 1. Neuroimaging data indicates increases in right side striatal-auditory cortical connectivity in patients exhibiting bilateral tinnitus (Hinkley et al., 2015) while tinnitus induction via unilateral noise exposure in mice is shown to cause bilateral hyperactivity in the IC (Hsiao and Galazyuk, 2021). We will therefore include three different microinjection targets: right CN, left CN (n=13-14 per side) and bilateral CN (n=13-14). SBAD testing will be completed one month post-surgery and compared to pre surgery SBAD scores.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1. A composition for treating a neurological disease or condition, the composition comprising: a vector, the vector comprising an adeno-associated virus (AAV) and a neuron specific promoter operatively coupled to a gene of interest.

2. A composition for treating a neurological disease or condition, the composition comprising: a first vector comprising a first AAV, and a first neuron specific promoter operatively coupled to a first gene of interest, and a second vector comprising a second AAV, and a second neuron specific promoter operatively coupled to a second gene of interest.

3. A composition comprising: a vector comprising a gene therapy construct, wherein the gene therapy construct comprises a heterologous nucleic acid encoding a designer receptor, wherein the designer receptor is configured to interact with a small molecule actuator, wherein the vector comprising the heterologous nucleic acid is configured to be expressed in a neuron, wherein expression of the heterologous nucleic acid modulates an inhibitory or an excitatory activity of the neuron, wherein the modulation results in a reduction of a symptom or presence of a disease or condition associated with the neuron.

4.-87. (canceled)

88. The composition of claim 1, wherein the gene of interest encodes a modified neuronal receptor.

89. The composition of claim 88, wherein the modified neuronal receptor is a modified muscarinic receptor.

90. The composition of claim 89, wherein the modified muscarinic receptor comprises a designer receptor exclusively activated by designer drugs (DREADD).

91. The composition of claim 90, wherein the DREADD comprises one or more of hM3Ds, hM1Dq, hMD5q, hM4Di, or hM2Di, or hM3Dq.

92. The composition of claim 1, wherein the neurological disease or condition comprises tinnitus, Meniere's disease, noise induced hearing loss (NIHL), sensorineural hearing loss, auditory neuropathy spectrum disorder, and/or central auditory processing disorder.

93. The composition of claim 2, wherein the first and/or second gene of interest encodes a modified neuronal receptor.

94. The composition of claim 93, wherein the modified neuronal receptor is a modified muscarinic receptor.

95. The composition of claim 94, wherein the modified muscarinic receptor comprises a designer receptor exclusively activated by designer drugs (DREADD).

96. The composition of claim 95, wherein the DREADD comprises one or more of hM3Ds, hM1Dq, hMD5q, hM4Di, or hM2Di, or hM3Dq.

97. The composition of claim 2, wherein the neurological disease or condition comprises tinnitus, Meniere's disease, noise induced hearing loss (NIHL), sensorineural hearing loss, auditory neuropathy spectrum disorder, and/or central auditory processing disorder.

98. The composition of claim 3, wherein the designer receptor comprises a designer receptor exclusively activated by designer drugs (DREADD).

99. The composition of claim 98, wherein the DREADD comprises one or more of hM3Ds, hM1Dq, hMD5q, hM4Di, or hM2Di, or hM3Dq.

100. The composition of claim 3, wherein the small molecule actuator comprises an atypical antipsychotic.

101. The composition of claim 100, wherein the atypical antipsychotic comprises Clozapine-N-oxide.

102. The composition of claim 3, wherein the symptom of the neurological disease or condition comprises vertigo, dizziness, hearing impairment, buzzing, clicking, humming, hissing, roaring, or ringing, or a combination thereof.

103. The composition of claim 3, wherein the neurological disease or condition comprises tinnitus, Meniere's disease, noise induced hearing loss (NIHL), sensorineural hearing loss, auditory neuropathy spectrum disorder, and/or central auditory processing disorder.


SEQUENCE LISTING (13 sequences)

SEQ ID NO: 1 - DNA, 1778 bp, Homo sapiens
  source 1..1778: mol_type = other DNA
ttcaaggcctgctcgggtgcgcgcttgtgaaaaatgaccgactgtctctgctggtactgctgcttgcgcctcttttttttgtcacactggcacagcagcagcatcttgaaagtggttctgaatgttttgttgcacagagcatagcacacggggttcacggtgctgttgatgtagcacagccagtagcccagattccaaaaggttttgggtatgcagctgtcacaaaaggtgttcaccagaaccatgatgttgtatggggtccaagtgatgatgaaggcaagcaagatcgcactgagggtctgggccgctttcttctccttgaccagggacatccttttccgcttagtgatctgacttctggtcttcagagcaaacctcttggccagagtggcttccttgaaggacagaggtagagtggccgtgctcttacccactgaggagttgacgtcagaagtcttagctgtgtccacggctgactctagctggatgggaagcttggagaagctttttggaaaactgcctccatcgtccacgctcttctgggcctgcagcttgtcggctttcctctccaagtccaccatccccagctcctcctcaggcacctgcaggttgtccgatgagggtaacttggtggagttgaggatggtgctgtgacccggaagcttgagcacgatggagtagatggctctcgtctcggagccaatgtcctcctcgtcggaggaggcggagttctccagggaggcagcagcatcattgttgttccaactgtcactgctgctgtggtcttggtccatctgctcggagctgggtttccagctcttggttgtgaaccagaagtggcagcggccatacttcctcctgttggagcgtttcatgctttgctgttgaagttcgtaactgctgcagcttcgagaactgcccgtggggtggacaaagttttctgtctctgcctctgtcccagaggcttgcaggccagcaagctctttggtacgcttttcagtttccttatagatcctccagtataaaatagtcataatggtgacaggcatataaaaaccagcgatggctgtgccaaaagtaatggtgggctcactgaggaactgaatgaagcactctcccggaggcacagttctctttccaacaaagtattgccagaacaagatggcaggagcccaaaggacaaaggagatgacccaagccagaccgatcatcacaccggctctctttgttgttcgtttggctcggtacgtgagcggcctcgtgatggaaaagtatctgtcaaagctgatgaccagaagattcataacagaggcattgctggctacgcagtcaatggcaagccagaggtcacaggccaagttccctaaggcccatcgattcatgatgatgtaggtcgtaaacagattcattgaaatgaccccgataatcagatcggcacaggccaggcttaagaggaagtagttgttgaccgtcttcagctgcttgttgaccttaaatgacacaattaccaggatgttgccgatgatggtcaccaaggccaggatgcccgttaagaaagcgatgaagaccacttgccagacggtatgacctcccagagggtcatcggtggtaccgtctggagaggagaaattgccagctgctcgagaaacattgtagctgccgaaatgagtgacggttcccgggggcagccctgcatcggaggggctgtgtatccaggaggagctgatgtttggaaacaaaggcgaggttgtactgttattgtgcaaggtcatggtggc

SEQ ID NO: 2 - DNA, 1293 bp, Homo sapiens
  source 1..1293: mol_type = other DNA
ttaacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 3 - DNA, 600 bp, Homo sapiens
  source 1..600: mol_type = other DNA
cgaaggatgggtgccagggaaactgacttatagacgacccgctccgcgagagactgacaactcagtcctgcgggctttcggggcgcccagaccagtccttagccagaggatctggggctgcacagctcccggaccctggagggggtggtccgggggtgggagactgcgagcatcagcccacgccggccccctcccaccctgcggcggcgaggtgggaagggccaggatgcgagcttagcacctcccctagaaatgcagcacttcgcccccccacctcccctgccctcggcgctccctctttcacgcgctccctcccgcctccttcccaagggcgcccttcttctgcccccagctcacgtctgaatccctcggcgccccctttctcttctcctagccccttcctcacgtcccctgcctccgggtatttcctctctccaatcccccacccccgcaccgcctgattccgaggggcgggagcgcattgggctgcgcacgggtgggggcgccgcgccagcttcgcgtagctgctctgacgccgctgccgccgccgccgccgccgccgccctccgcagcccagctcgcgccccgcggcagctccgc

SEQ ID NO: 4 - DNA, 416 bp, Homo sapiens
  source 1..416: mol_type = other DNA
ggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 5 - DNA, 600 bp, Homo sapiens
  source 1..600: mol_type = other DNA
gacccttaaagagaggagggttaggagtcagggagaggaaaagggacccaaggggcctgggaccagctgagaaagacttaggaggccagaagagtaagtgaaaagaattggggtggcaggcagaggagttggtggggggtggggcagccatacctgacacagagtgaagtcggctaggaaaggacaggtgtgggtgcatggtaggggctgcaggggaaagttggtggtgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtattttt

SEQ ID NO: 6 - DNA, 600 bp, Homo sapiens
  source 1..600: mol_type = other DNA
tcagcctcctgtctgccgaccgggcacctgcttgcccaatgaatatcttgctcactagcctggtgcttctgaggtcaggaccacgtcttgctactgtcccctgcacccagtacagtgttagaatgtagtaaatgctcagtatgcttgttcaatgaatgaaggttgactgaatcaaccacagagatgatcagagtctcctttacagatggggaaactgaggcactgagaaatgatctccctgggtgccatggcaaggggaggccgccacccgacatgggcatcgggaaagtggggccgaggccactctttgacaaaagtgactcagcagctggcggctggcgtgggctgtggaatttctaagcatcccctcacatcctgagcgaacgggcgatgggagggagtaagcagagtgaaaagaagaaaaacaagaaacttgggggaaaagaaagagagagggggaaaaaaactgaaattgaaaacagacgcacgcgtccaccctccctgccccgccgcccccccttctcccagctgggccaatcagaagccaccctgcagcctttcccccaaagtggcgggggaagtgggggggcatggaagccc

SEQ ID NO: 7 - DNA, 690 bp, Homo sapiens
  source 1..690: mol_type = other DNA
gatctaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcgggg

SEQ ID NO: 8 - DNA, 600 bp, Homo sapiens
  source 1..600: mol_type = other DNA
ggggcaccggctggagtccggcgttgaccgcagccgggacgcgcccgccacctgggcggggaaggagtgagtcccctcggggcgcagcggcgcgcgctggcccggatcccagctgcgccgcgccctccctgcgagcggtgagtacaggcgcccacgggcatctccaaccctagttaggggacacttggagacaacgcaggtcggacgtccgccggccgttctaggagttggtgtgcggggaatggggggtgctcccgctcgcttagggctttgggtccggctaggggttgaccccaagaggcgctggatgtcggggctgagctttgctttcaggggactcaagcttacttgaggttctgacattggatttgcctcgcgggccctgcgcgtgctgcgcggggatccggagactggcgaggtaaccagggagggcaagcactcaccggggcgtcctccgcccacctcgcccgcagaaccatccagggggaccacgtgcggctgtcgcccgcaactctgcctgtcaagcgaggaccgcccccagggcaggggaggggacgcgcgggcggggtgggctgtgccccgcgggaaccccgccggcct

SEQ ID NO: 9 - DNA, 892 bp, Homo sapiens
  source 1..892: mol_type = other DNA
gtgcgggatcaggggcctggttttctttctgtgtgtgacttctgaatctgacacagaatctggcaaactgaggacagttggtaactaattgctgcagaagaaagaaaagaaagagaaagaaagaaaaagaaaggaaggaagaaagaaagaaagaaaggaaagaaagaaagagaaagaaagaaagaaagaaagaaagaaagaaagaaagaaagaaagaaagaaagaaaagaaaagaaaagaaagaaaaggagagaggaagaaaaggggagaagaacgaggttcctctgcagcccaaatgacagtgggcagggtagcagcggaacacccttcaccactctatctctttctccctctccccactccctgcctcacacacctcagtttggagtcaagggtgagctggaacccctgaggcctccttttgcatttccatgtcaggtcaactccaaacaggctttttcctttggatgagtccagcaatagaactgactcatagattccaagacctgaagtcagaaaacggatgcggacctcttccaacacctcctctccttgcctgacttgctggatgggtggtgcctcccgcaacccttggcttctgagtcctcaaaggagaagactggcgagcagacggtgaggacccagcctgcaatcacagcttattactctgggtgtgggtgggagcgcagtgggcggtcgagggttgcgttcccgcctcaaaacaagggatggcggaatcccccaacccctcctacccgttcaggccggggatcgccgaggaggtacagctcctttggtggggggcgggggcggggcctgtctcaggggcggggaccggggcacctccctcccgcgctccccgcgctcgggcgccgcagagctgtccagcttc

SEQ ID NO: 10 - DNA, 1300 bp, Homo sapiens
  source 1..1300: mol_type = other DNA
aagggacttgtctgagaatatgcggaacatttccttctaccaggcactagatcctcgcacaaaagtgctgagtccgctcccaaccccaggcccgtggctttgagcagcaggtcacttaacatctaacgtctttatgctgtttcctcatctgtgcgagagcagctatgtctacctggcaaggctgtagtgagagatcatatcagcataggaatggggctcagccccatgcacagaggacagttcttgtttcattcttttccttgctgtttctcttcctttcctggcagaatatggaggaaggaagcatcgctgccatctacagtggtcacggaaggcttcatggaagaggcgagccctgcctgggcctcaattttgggtgctggagggaagcaggggccaagagttattaatagtcttggcctgatgggcccagggaggctgaatgtgatacagacacccagcaccacggttggggagtacctgacaccggaaggggagggggccggggctacggggagtgccacctcccaaaatagccagagcagaagcctatataggtggccatcccacctccaggctcacttcccgacaggacttcccaccagcccagcctttcagtgcaggctccagccctccacccccacccgaggtgagtggcagctaccgaggttggaggatagagggatgcagcaggatgagccagctggaagggagagctacatctcccctgtccgtagtgacccggggaggggggtgcggtgggggtgctggaggcagggcagctgtggaatgtagggctgagagcatgcattcctgcttctccacccagactcctggtgtggcctggggcaaactccccccacccaccgccccccgtggctgggcctcagattccccagccttagaacgagagggctggagtcagacatctgtggactctggtgatgccatcagagaggacccagagggcagaggaggactacccagggctgatgggctgacctaatgacattgatgctctggctgggagggctcagtcccggagggggtgtcaagttgataggagaactcagcagcagggagagatggaggaaggagatggaagaagagtgggcccctgttgaggctgcagcctttgccacagggtagctgcttaggggctcaccaggagccagtgtgtggctgagatgtatgggacctgatagagctgggtcccaggcaccagggaggatcacccagagagtcactgaactccacacgagtgagtccaggggtgtaccctgctctgctattgctttac

SEQ ID NO: 11 - DNA, 600 bp, Homo sapiens
  source 1..600: mol_type = other DNA
cactttctaattacctaacaaagtaatttgggattcaccgtggggctggcgggatggggaaccaacatccacttacagccagggtggtgtacgccacaccgcgagggactggctgtattggtagtagtggcgatggggtgggggctcttgtgcacttttttgtacctagcaccccagaagacagaaggagcagcggcagatcttgcctccactagccctgccccctaggcctggtggcgcgcgctttggccactggaggtgaggaagatcctgggtggcaacgagagacgtctacacatagataagggagcctaccacaggcctgggtttgctgcgggtttcggtgtcccccagggactcctaaactctggaagtcgcgctagacctcaggaggccggaaaatgaccacctggcgtcggtggctccagcagatctgagcccctaggccccctgggctccacgctgagccgcgctctcggggcgagtgagcccggggctctccgcgcatggggagtggagaagggagggggaggaggaggaaggctgggagggaaagaggatgtgtgtgttgggggagggggcggtgcagccgcctttcct

SEQ ID NO: 12 - DNA, 587 bp, unidentified
  source 1..587: mol_type = other DNA
tgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccg

SEQ ID NO: 13 - DNA, 1439 bp, synthetic construct
  source 1..1439: mol_type = other DNA
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggcg